(19) United States
(12) Patent Application Publication
(10) Pub. No.: US 2021/0371782 A1
(43) Pub. Date: Dec. 2, 2021

(54) METHODS FOR OPTICAL MICROPATTERNING OF HYDROGELS AND USES THEREOF

(71) Applicant: President and Fellows of Harvard College, Cambridge, MA (US)

(72) Inventors: Kevin Kit Parker, Cambridge, MA (US); Janna C. Nawroth, Boston, MA (US); Lisa Scudder, Belmont, MA (US)

(21) Appl. No.: 16/322,507

(22) PCT Filed: Aug. 4, 2017
(86) PCT No.: PCT/US17/45442

§ 371 Date: Feb. 1, 2019

Related U.S. Application Data
(60) Provisional application No. 62/371,385, filed on Aug. 5, 2016.

(51) Int. Cl. C12M 3/00; B23K 26/352; B23K 26/00; C12M 1/00; C12M 3/06; C12N 5/00

(52) U.S. Cl. CPC C12M 21/08; B23K 26/355; B23K 26/0006; B23K 2103/30; C12M 23/16; C12N 5/0075; C12M 23/20

(57) ABSTRACT

The present invention provides methods for optically micropatterning hydrogels, which may be used for, e.g., regenerative medicine, synthetic or cultured foods, and in devices suitable for use in high throughput drug screening assays.

### RELATED APPLICATIONS

This application claims the benefit of priority to U.S. Provisional Patent Application No. 62/371,385, filed on Aug. 5, 2016, the entire contents of which are incorporated herein by reference.

### GOVERNMENT SUPPORT

This invention was made with government support under grant number W911NF-12-2-0036, awarded by the Defense Advanced Research Projects Agency (DARPA); and under grant number 4UH3TR000522, awarded by the National Institutes of Health (NIH). The government has certain rights in the invention.

### FIELD OF THE INVENTION

The present invention provides methods for optically micropatterning hydrogels, which may be used for, e.g., regenerative medicine, synthetic or cultured foods, and in devices suitable for use in high throughput drug screening assays.

### BACKGROUND OF THE INVENTION

Identification and evaluation of new therapeutic agents and identification of suspect disease associated targets typically employ animal models which are expensive, time consuming, require skilled animal-trained staff and utilize large numbers of animals. In vitro alternatives have relied on the use of conventional cell culture systems that are limited in that they do not allow the three-dimensional interactions that occur between cells and their surrounding tissue. This is a considerable disadvantage as such interactions are well documented as having a significant influence on the growth and activity of cells in vivo since in vivo cells divide and interconnect in the formation of complex biological systems creating structure-function hierarchies that range from the nanometer to meter scales.

Similarly, efforts to build biosynthetic materials or engineered tissues that recapitulate these structure-function relationships often fail because of the inability to replicate the in vivo conditions that coax this behavior from ensembles of cells. For example, engineering a functional muscle tissue requires that the sarcomere and myofibrillogenesis be controlled at the micron length scale, while cellular alignment and formation of the continuous tissue require organizational cues over the millimeter to centimeter length scale. Thus, to build a functional biosynthetic material, the biotic-abiotic interface must contain the chemical and mechanical properties that support multiscale coupling.

Current methods to recapitulate the in vivo environment of cells and tissues in vitro are encumbered by several limitations, including labor intensiveness, imprecise alignment of tissue, e.g., muscle fibers, and low throughput. For example, micromolding and soft lithography have been used. In soft lithography techniques, PDMS stamps for microcontact printing are generated in order to provide the appropriate cues for cell adhesion and tissue morphogenesis. Such methods, however, are costly and slow, and depend on the use of toxic chemicals and photoresists requiring cleanrooms and fume hoods to complete. Soft lithography also requires photomasks that are costly and require proper alignment by the user.

Accordingly, there is a need in the art for improved methods and systems that are less expensive, time efficient, reproducible, and that permit cell adhesion and tissue morphogenesis in order to recapitulate in vivo structure-function hierarchies.

### SUMMARY OF THE INVENTION

The present invention is based, at least in part, on the discovery of agile manufacturing methods for micropatterning of hydrogels that may be used for, e.g., tissue engineering and fluidic device applications. The methods of the present invention reduce process time by more than half and achieve a much higher throughput in comparison with previous methods. For example, the micromolding process for micropatterning hydrogels requires at least 6-8 days for completion, and requires at least 13.5 man-hours. The optical patterning methods for micropatterning hydrogels described herein, however, surprisingly can be completed within 2 days' time, and require less than half of the man-hours required by the micromolding methods. In addition, the methods of the invention do not rely on toxic chemicals, thus, eliminating the need for a cleanroom used in soft lithography, eliminate the use of silicon wafers, and offer fine control over patterning and cutting/ablation of a hydrogel, thereby increasing reproducibility and eliminating user error that may occur by imprecise alignment of photomasks. Furthermore, the methods of the invention are cell safe, guide cell development into forming tissues, e.g., anisotropic (aligned) tissues, allow for single cell micropatterning, do not significantly alter surface properties of the hydrogel, e.g., stiffness, and can be used for, e.g., microfluidic technologies including, for example, muscle thin film technologies, such as drug screening.

Accordingly, in one aspect, the present invention provides methods for optical micropatterning of a surface of a hydrogel that may be used for, e.g., regenerative medicine or in a fluidic device for, e.g., drug screening, e.g., high-throughput drug screening. The methods include providing a base comprising a cyclic olefin copolymer (COC), wherein a surface energy of at least a portion of a surface of the base is modified; forming a hydrogel layer on the surface of the base overlying the portion of the surface having the modified surface energy, the hydrogel layer being susceptible to cross-linking by exposure to light, the hydrogel layer having a surface facing away from the base, wherein the modification of the surface energy of the portion of the surface of the base promotes adhesion of the hydrogel layer to the surface of the base; and exposing at least a portion of the hydrogel layer to light in a preselected pattern, thereby optically micropatterning the surface of the hydrogel layer.

The devices produced by the disclosed methods, e.g., fluidic devices, may comprise a solid support structure as a base and a micropatterned hydrogel layer configured to support growth of a functional tissue, e.g., a functional muscle tissue. In some embodiments, a functional muscle tissue is disposed on the micropatterned surface of the hydrogel layer.

In some embodiments, the methods of the invention include modifying a surface energy of at least a portion of a surface of a base comprising a cyclic olefin copolymer (COC); forming a hydrogel layer on the surface of the base overlying the portion of the surface having the modified surface energy; and exposing at least a portion of the hydrogel layer to light in a preselected pattern, thereby micropatterning the surface of the hydrogel layer. In an exemplary embodiment, the hydrogel layer is susceptible to cross-linking by exposure to light. The method may also include modifying the surface energy of the portion of the surface of the base to promote adhesion of the hydrogel layer to the surface of the base.

In a specific embodiment, the surface energy of at least the portion of the surface of the base is modified by plasma treatment.

In one embodiment, the preselected pattern is anisotropic. Alternatively, the preselected pattern can be any desired shape, such as a geometric shape, e.g., a square saw-tooth pattern, a rectangle, a square, a circle, a triangle, etc.

In another embodiment, the pre-selected pattern includes a plurality of lines or a plurality of line segments with a peak-to-peak line separation in a range of about 1 μm to about 100 μm. In one embodiment, the peak-to-peak line separation is about 10 μm to about 30 μm. In another embodiment, the peak-to-peak line separation is about 15 μm or about 20 μm.

In one, a peak-to-trough height of the resulting micropattern in the surface of the hydrogel layer falls in a range of about 0.5 μm to about 10 μm. In one embodiment, the peak-to-trough height is about 1 μm to about 5 μm. In another embodiment, the peak-to-trough height is about 2 μm or about 3 μm.

In one embodiment, a laser is used to expose the portion of the hydrogel layer to light in the preselected pattern. In one embodiment, the laser has a beam diameter in a range of about 10 μm to about 20 μm. In another embodiment, the beam diameter is about 20 μm.

In one embodiment, the speed of the laser when serially writing falls in a range of about 0.0005 W/mm/s to about 0.003 W/mm/s. In one embodiment, the speed of the laser is about 0.0009 W/mm/s to 0.001 W/mm/s.

In one embodiment, the laser is a microlaser.

In one embodiment, the laser light is ultraviolet (UV) light. In another embodiment, the laser light is visible light. In one embodiment, the wavelength of the light is about 300 nm to about 500 nm. In a preferred embodiment, the wavelength of the light is about 300 nm to about 400 nm, about 400 nm to about 450 nm, or about 450 nm to about 500 nm. In another embodiment, the wavelength of the light is about 315 nm to about 380 nm.

In one embodiment, the method further includes forming the hydrogel layer on the surface of the base overlying the portion of the surface having the modified surface energy by depositing an aqueous solution comprising gelatin on the surface of the base. In one embodiment, the aqueous solution further comprises transglutaminase. In one embodiment, the aqueous solution comprises about 5 to about 20% w/v gelatin and about 4% or more w/v transglutaminase. In another embodiment, the aqueous solution comprises about 9 to about 10% w/v gelatin and about 4% w/v transglutaminase. In yet another embodiment, the aqueous solution comprises about 10% w/v hydrogel and about 4% w/v transglutaminase.

In one embodiment, the method further includes forming the hydrogel layer on the surface of the base overlying the portion of the surface having the modified surface energy by further curing the deposited aqueous solution resulting in a cured layer. In one embodiment, the slide is cured for at least about 10 hours. In another embodiment, the slide is cured for at least about 24 hours. In still another embodiment, the slide is cured for up to about one month.

In one embodiment, the method further includes forming the gelatin layer on the surface of the base overlying the portion of the surface having the modified surface energy by further treating the cured layer with a second solution that makes the cured layer susceptible to cross-linking by exposure to UV light. In one embodiment, the second solution comprises riboflavin-5′ phosphate, Rose Bengal, or SU-8 Photoresist. In one embodiment, the second solution comprises riboflavin-5′ phosphate. In one embodiment, the second solution comprises about 0.01% w/v to about 0.3% w/v riboflavin-5′ phosphate.

In one embodiment, the method further includes rinsing the cured later in an aqueous solution, e.g., water, following treatment with the second solution.

In another embodiment, the cured layer is hydrated in an aqueous solution, e.g., water, prior to treating the cured layer with the second solution, e.g., to facilitate removal of a casting surface. In one embodiment, the cured layer is hydrated for at least about 10 hours. In another embodiment, the cured layer is hydrated for at least about 3 hours for each centimeter of the maximal radius of the cast hydrogel.

In yet another embodiment, the curing occurs in a humidified chamber of greater than about 90% relative humidity, e.g., the cured layer does not require rehydration to facilitate removal of a surface of the base.

In still another embodiment, the method further comprises masking a portion of the surface of the base using an adhesive mask prior to modifying the surface energy of at least a portion of the base such that the surface energy of the masked portion of the surface of the base is not modified during the modification of the surface energy of at least a portion of the surface of the base. Subsequently, the adhesive mask may be removed from the surface of the base after hydration of the cured layer.

In one embodiment, the method further includes rinsing the micropatterned hydrogel layer with an aqueous solution, e.g., water.

In another embodiment, the method further includes cutting through a full thickness of the hydrogel layer using a laser after the surface of the hydrogel layer has been micropatterned. In a preferred embodiment, the laser is a UV laser.

In one embodiment, the method further includes ablating a portion of the hydrogel layer using a laser after the surface of the hydrogel layer has been micropatterned. In one embodiment, the laser is a UV laser.

In still another embodiment, the method further includes modifying a surface energy of a portion of the surface of the base surrounding the micropatterned hydrogel layer to inhibit cell adhesion to the surface of the base. In a specific embodiment, the surface energy of the portion of the surface of the base surrounding the micropatterned hydrogel layer is modified using a laser. In one embodiment, the laser is a UV laser.

In one embodiment, the method further includes seeding the micropatterned surface of the hydrogel layer with cells, e.g., muscle cells.

Additional features and advantages are realized through the techniques of the present disclosure. Other embodiments and aspects of the disclosure are described in detail herein and are considered part of the invention. The recitation herein of desirable objects, which are met by various embodiments of the present disclosure, is not meant to imply or suggest that any or all of these objects are present as essential features, either individually or collectively, in the most general embodiment of the present disclosure, or in any of its more specific embodiments.

### BRIEF DESCRIPTION OF THE DRAWINGS

The features and advantages of the present disclosure will be more fully understood from the following description of exemplary embodiments when read together with the accompanying drawings, in which:

FIGS. 1A-1F depict a method for optical micropatterning of a hydrogel layer in accordance with one embodiment of the invention.

FIG. 1A depicts a hydrogel (e.g., gelatin) crosslinked with microbial transglutaminase and cured. Inset shows stereomicroscope image of cured gelatin hydrogel. Scale bar is 50 μm.

FIG. 1B depicts line patterns that are written into the hydrogel using a UV laser with a wavelength of 355 nm after the addition riboflavin-5′phosphate to the hydrogel.

FIG. 1C depicts the hydrogel in a hydrated state with the UV laser micropatterned lines corresponding to a micropatterned variation in height of the top surface of the hydrogel. Inset shows stereomicroscope image of top surface of UV laser micropatterned gelatin. Scale bar is 50 μm.

FIG. 1D depicts the addition of 0.05% riboflavin-5′phosphate to the gelatin surface.

FIG. 1E depicts the UV laser etching of gelatin surface. Scale bar is 1 cm.

FIG. 1F shows the untreated gelatin hydrogels cannot be effectively micropatterned with the UV laser engraver and instead exhibit burn marks and bubbles. Scale bar is 50 μm

FIG. 2 is a schematic showing an exemplary method for optical micropatterning of a hydrogel (e.g., gelatin) layer in accordance with one embodiment of the invention.

FIGS. 3A-3E(ii) depict measurements of surface topography and nanomechanics of micromolded and UV laser micropatterned hydrogels.

FIG. 3A is a graph depicting atomic force microscopy of micromolded hydrogel topography over a 40 μm (x)×40 μm area (y). Z-axis scale ranges from 2.50 (top) to −2.50 μm (bottom).

FIG. 3B is a graph depicting atomic force microscopy of UV laser micropatterned hydrogel topography over a 40 μm (x)×40 μm area (y). Z-axis scale ranges from 2.50 μm (top) to −2.50 μm (bottom).

FIG. 3C is a box plot depicting the elastic modulus for micromolded hydrogels (MM), UV laser micropatterned hydrogels (UV), and unpatterned hydrogels at 2.50 μm (top) and −2.50 μm (bottom) for patterned gels from force distance curves (n=64-96, 3 different areas). Gray line indicates the mean, black line in center indicates the median.

FIG. 3D(i) depicts a height map of micromolded hydrogel over a 40 μm (x)×40 μm (y) area. Gray line indicates cross-section of height map for Z-sensor cross-sectional distance. Dots indicate maximum and minimum points for the Z-axis.

FIG. 3D(ii) depicts the Z-sensor cross-sectional distance of UV micromolded hydrogel in FIG. 3D(i) Gray line indicates cross-section of height map and dots indicate maximum and minimum points for the Z-axis.

FIG. 3E(i) depicts a height map of UV laser micropatterned hydrogel over a 40 μm (x)×40 μm (y) area. Gray line indicates cross-section of height map for Z-sensor cross-sectional distance. Dots indicate maximum and minimum points for the Z-axis.

FIG. 3E(ii) depicts the Z-sensor cross-sectional distance of UV micromolded hydrogel in FIG. 3E(i) Gray line indicates cross-section of height map and dots indicate maximum and minimum points for the Z-axis.

FIGS. 4A-4D depict the surface topography and nanomechanics of UV micropatterned pillars for single cell islands.

FIG. 4A depicts atomic force microscopy of micropatterned pillar topography over a 40 μm (x)×40 μm area (y). Z-axis scale ranges from 2.50 μm (top) to −2.50 μm (bottom).

FIG. 4B is a box plot of the elastic modulus for UV laser micropatterned hydrogels with pillar topography at 2.00 μm (top) and −2.00 μm (bottom) for patterned gels from force distance curves (n=84-95, 3 different areas). Gray line indicates the mean, black line in center indicates the median.

FIG. 4C is a height map of UV micropatterned pillars over a 40 μm (x)×40 μm (y) area. Gray line indicates cross-section of height map for Z-sensor cross-sectional distance

FIG. 4D is the Z-sensor cross-sectional distance of UV micropatterned pillars. Gray line indicates cross-section of height map and gray dots indicate maximum and minimum points for the Z-axis.

FIGS. 5A-5C depict engineered functional anisotropic cardiac tissue grown on UV laser micropatterned hydrogels.

FIG. 5A is a brightfield image of neonatal rat ventricular myocytes (NRVMS) seeded on UV laser micropatterned hydrogels. Scale is 50 μm.

FIG. 5B is immunohistochemistry of NRVMs seeded on UV laser micropatterned hydrogels. Light gray: chromatin, dark gray: α-actinin. Scale is 50 μm.

FIG. 5C is a box plot of orientational order parameter (OOP) of sarcomeric α-actinin between micromolded (MM) and UV laser micropatterned gelatin (UV). N=3-5 slides, 29-33 images. The gray line represents the mean, black center line represents the median, and error bars represent SEM.

FIGS. 6A-6D depict engineered functional anisotropic muscle tissue strips fabricated on UV laser micropatterned hydrogels for, e.g., heart-on-a-chip applications

FIG. 6A schematically depicts anisotropic functional muscle tissue strips (also referred to as muscle thin films, or MTFs) fabricated using UV laser micropatterning of hydrogels.

FIG. 6B(i) is a stereoscope brightfield image of engineered NRVM cardiac muscular thin films in diastole after 5 days in culture. Gray line indicates height of MTF detected by tracking software. Boxes represent initial length. Scale bar is 0.5 mm.

FIG. 6B(ii) is a stereoscope brightfield image of engineered NRVM cardiac muscular thin films systole after 5 days in culture. Gray line indicates height of MTF detected by tracking software. Boxes represent initial length. Scale bar is 0.5 mm.

FIG. 6B(iii) is a graph of the raw contractile stress traces at 0, 1, and 2 Hz pacing frequencies for the same representative MTF in B(i) and B(ii)

FIG. 6B(iv) is a graph of the contractile stress of UV laser micropatterned muscular thin films (n=9-13 films, 5-6 heart chips). Bars represent the mean±SEM for diastolic (black), systolic (white), and twitch stress (gray).

FIG. 6C is a graph depicting the beat rate of engineered MM (black) and UV-M (gray) NRVM cardiac tissues in culture over a 27 day period in beats per second.

FIG. 6D is a graph depicting the contractile stress of UV-M muscular thin films after 27 days in culture (n=2-3 films, 1 heart chip). Bars represent the mean±SEM for diastolic (black), systolic (white), and twitch stress (gray). *P<0.05 compared to 1 Hz pacing by Student's T-Test.

FIGS. 7A-7C depict images of and measured contractile stress data from micromolded gelatin functional muscle tissue strips.

FIG. 7A depicts stereomicroscope image of 10 μm by 10 μm micromolded line patterns. Scale=0.5 mm.

FIG. 7B(i) depicts a micromolded gelatin functional muscle tissue strip in diastole. Gray bar indicates thin film cantilever height. Scale=0.5 mm.

FIG. 7B(ii) depicts a micromolded gelatin functional muscle tissue strip in systole. Gray bar indicates thin film cantilever height. Scale=0.5 mm.

FIG. 7C depicts measured micromolded gelatin functional muscle tissue strip contractile stress calculated using elastic modulus value measured from atomic force microscopy (107 kPa). Mean Stress ±SEM for diastolic stress is indicated by the black bars, systolic stress is indicated by the white bars, and twitch stress is indicated by the gray bars at increasing pacing rates. *P<0.05 compared to spontaneous contractile stress by one way ANOVA.

FIG. 8A-8F(ii) depict applications of optically patterned hydrogels for human iPS-cardiomyocyte tissue engineering.

FIG. 8A is a brightfield image of human iPS-derived cardiomyocytes (hiPSCs) seeded on UV laser patterned gelatin. Scale bar is 50 μm.

FIG. 8B is an immunostained image of hiPSCs seeded on UV laser patterned gelatin. White: a-actinin, light gray: chromatin. Scale bar is 20 μm.

FIG. 8C is a stereoscope image of UV laser patterned micropillars. Scale bar is 50 μm.

FIG. 8D is an atomic force microscopy image of hydrated micropillars over a 40 μm (x)×40 μm (y) area. Z-axis ranges from 2 to −2 μm.

FIG. 8E is a brightfield image of hiPSCs seeded on UV laser patterned micropillars. Scale bar is 50 μm.

FIG. 8F(i) is an immunostained image of hiPSCs on single cell islands that maintained circular morphologies. Merge image shows light gray: actin, white: a-actinin, medium gray: chromatin. Scale bars are 10 μm.

FIG. 8F(ii) is an immunostained image of hiPSCs on single cell islands that spread out over the islands. Merge image shows light gray: actin, white: a-actinin, medium gray: chromatin. Scale bars are 10 μm.

FIGS. 9A-9E compare hydrogel patterning and adhesion achieved under various conditions.

FIG. 9A is an image of micromolded gelatin.

FIG. 9B demonstrates that when no riboflavin is used with a glass base the gelatin adheres to the base, but the gelatin burns and/or boils, and cannot be patterning.

FIG. 9C demonstrates that when riboflavin is used on a polycarbonate base, the gelatin adheres, but the gelatin burns and/or boils, and cannot be patterned.

FIG. 9D demonstrates that when riboflavin is used on an acrylic base, patterning occurs, but the gelatin does not adhere to the base.

FIG. 9E shows the UV-patterned gelatin on a COC base in accordance with some embodiments of the invention.

FIGS. 10A-B show the effect of riboflavin and riboflavin concentration on micropatterning. For example, several different types of riboflavin have been tested. It was determined that riboflavin 5′ phosphate is most soluble in water, and ideal for patterning gels at 0.05% w/v concentration.

FIG. 10A is a hydrogel comprising gelatin treated with 0.1% w/v riboflavin 5′-phosphate; on a COC modified base. The hydrogel was optically patterned using a microlayer (laser power=0.16 W, frequency=50 kHz, speed=80 mm/s).

FIG. 10B is a hydrogel comprising gelatin treated with 0.05% w/v riboflavin 5′-phosphate; on a COC modified base. The hydrogel was optically patterned using a microlayer (laser power=0.16 W, frequency=50 kHz, speed=80 mm/s).

FIGS. 10C-10D show the effect of laser speed on micropatterning. For example, patterning too slow causes wavy lines and bubbles, and sometimes burning (FIG. 10C).

Patterning too fast does not produce lines.

FIG. 10C is a hydrogel comprising gelatin treated with 0.05% w/v riboflavin 5′-phosphate; on a COC modified base. The hydrogel was optically patterned using a microlayer (laser power=0.13 W, frequency=50 kHz, speed=120 mm/s).

FIG. 10D is a hydrogel comprising gelatin treated with 0.05% w/v riboflavin 5′-phosphate; on a COC modified base. The hydrogel was optically patterned using a microlayer (laser power=0.13 W, frequency=50 kHz, speed=135 mm/s).

FIGS. 11A-11C show neonatal rat ventricular myocyctes (NRVMs) seeded and cultured on hydrogels produced by the methods of the invention and demonstrate that the hydrogels are biocompatible and as effective as traditional micromolding as can be seen by measuring Orientational Order Parameter (OOP) of sarcomere alignment (0=disorder, 1=perfect order).

FIG. 11A shows neonatal rat ventricular myocyctes (NRVMs) seeded and cultured on hydrogels comprising an isotropic micropattern produced by micromolding.

FIG. 11B shows neonatal rat ventricular myocyctes (NRVMs) seeded and cultured on hydrogels comprising an anisotropic micropattern produced by micromolding.

FIG. 11C shows neonatal rat ventricular myocyctes (NRVMs) seeded and cultured on hydrogels comprising an anisotropic micropattern produced by the methods of the invention.

FIG. 12A-D depict the effect of plastic carrier and riboflavin concentration on UV laser micropatterning. Scale bar is 7.5 mm.

FIG. 12A depicts a schematic of riboflavin application to cured gelatin to fabricate UV laser micropatterns. Inset: Image of riboflavin solution added to cured gelatin. Scale bar is 7.5 mm.

FIG. 12B depicts UV-M gelatin on Zeonor polymer carrier incubated with 0.05% riboflavin (w/v) solution for 10 minutes. UV laser power is 0.16 W, frequency is 50 kHz, speed is 80 mm/second. Scale bar is 50 μm.

FIG. 12C depicts UV-M gelatin on Topas polymer carrier incubated with 0.1% riboflavin (w/v) solution for 10 minutes. UV laser power is 0.16 W, frequency is 50 kHz, speed is 80 mm/second. Scale bar is 50 um.

FIG. 12D depicts UV-M gelatin on Topas polymer carrier incubated with 0.05% riboflavin (w/v) solution for 10 minutes. UV laser power is 0.16 W, frequency is 50 kHz, speed is 80 mm/second. Scale bar is 50 μm.

FIG. 13A(i)-C(ii) depict the contact mode atomic force microscopy images of molded and UV micropatterned hydrogel height.

FIG. 13A(i) depicts the atomic force microscopy topography images of MM gelatin.

FIG. 13A(ii) depicts corresponding step-height profiles displayed by the lines and the height change between locations indicated by dots of the atomic force microscopy topography images of MM gelatin in FIG. 13A(i).

FIG. 13B(i) depicts the atomic force microscopy topography images of UV-M gelatin

FIG. 13B(ii) depicts the corresponding step-height profiles displayed by the lines and the height change between locations indicated by dots of the atomic force microscopy topography images of UV-M gelatin in FIG. 13B(i).

FIG. 13C(i) depicts the atomic force microscopy topography images of UV-μPillars gelatin.

FIG. 13C(ii) depicts the corresponding step-height profiles displayed by the lines and the height change between locations indicated by dots of the atomic force microscopy topography images of UV-μPillars in FIG. 13C(i).

FIG. 14 depicts the atomic force microscopy topography of UN gelatin in liquid contact on a 20 μm2 area and Z-axis range of 300 nm.

FIG. 15A-H depict the micromechanics of molded and UV laser micropatterned hydrogels.

FIG. 15A depicts a brightfield image of micromolded (MM) gelatin lines. Scale is 50 μm.

FIG. 15B depicts a brightfield images of a UV micropatterned (UV-M) lines. Scale is 50 μm.

FIG. 15C depicts a brightfield images of a UV micropatterned square pillars (UV-μP). Scale is 50 μm.

FIG. 15D depicts a contact-mode AFM topography image in 3D for MM gelatin in liquid over an area of 40 μm² with a Z-sensor height range of 5 μm.

FIG. 15E depicts a contact-mode AFM topography image in 3D for UV-M gelatin in liquid over an area of 40 μm² with a Z-sensor height range of 5 μm.

FIG. 15F depicts a contact-mode AFM topography image in 3D for UV-μP gelatin in liquid over an area of 40 μm² with a Z-sensor height range of 5 μm.

FIG. 15G is a box plot showing the differences in maximum and minimum Z-sensor heights (AZ-sensor height) for MM, UV-M, and UV-μP gelatin (n=6-13, 2-4 samples each). *P<0.05 compared to MM gelatin by Kruskal-Wallis One Way ANOVA.

FIG. 15H is a box and whisker plot of elastic moduli of UN, MM, UV-M, and UV-μP where a minimum of n=75 FDCs were used for each Z-level of the pattern. The gray line represents the mean, black center line represents the median, and error bars represent the 5th and 95^{th} percentile. *P<0.05 compared to UN gelatin by Kruskal-Wallis One Way ANOVA.

FIGS. 16A-H depicts cardiac tissue engineering of neonatal rat ventricular myocytes and human iPSCs with UV laser micropatterning.

FIG. 16A shows the immunohistochemistry of NRVMs seeded on unpatterned (UN) gelatin after 5 days in culture. Light gray: chromatin, dark gray: α-actinin. Scale is 50 μm.

FIG. 16B shows the immunohistochemistry of NRVMs seeded on MM gelatin after 5 days in culture. Light gray: chromatin, dark gray: α-actinin. Scale is 50 μm.

FIG. 16C shows the immunohistochemistry of NRVMs seeded on UV-M gelatin lines after 5 days in culture. Light gray: chromatin, dark gray: α-actinin. Scale is 50 μm.

FIG. 16D is a box plot of orientational order parameter (OOP) of sarcomeric α-actinin between tissues engineered on UN (n=3 slides, 8 images) MM (n=4 slides, 24 images), and UV-M gels (n=4 slides, 44 images). The gray line represents the mean, black center line represents the median, and bars represent 5^{th} and 95^{th} percentiles. *P<0.05 vs. UN gelatin by Kruskal-Wallis one way ANOVA and Dunn's Test.

FIG. 16E is an image of immunostained human iPSC tissues engineered on MM lines. Light gray: chromatin, dark gray: α-actinin. Scale is 25 μm.

FIG. 16F is an image of immunostained human iPSC tissues engineered on UV-M lines. Light gray: chromatin, dark gray: α-actinin. Scale is 25 μm.

FIG. 16G is an immunostained image of a single compact iPSC on a UV μ-pillar island after 9 days in culture. Light gray: α-actinin, dark gray: chromatin. Scale bar is 10 μm.

FIG. 16H is an immunostained image of a single iPSC spread beyond the UV μ-pillar island. Light gray: α-actinin, dark gray: chromatin. Scale bar is 10 μm.

### DETAILED DESCRIPTION

The present invention is based, at least in part, on the discovery of agile manufacturing methods for micropatterning of hydrogels that may be used for, e.g., tissue engineering and fluidic device applications. The methods of the present invention reduce process time by more than half and achieve a much higher throughput in comparison with previous methods. For example, the micromolding process requires at least 6-8 days for completion, and requires at least 13.5 man-hours. The optical patterning methods described herein, however, surprisingly, can be completed within 2 days' time, and require less than half of the man-hours required by the micromolding methods. In addition, the methods of the invention do not rely on toxic chemicals, thus, eliminating the need for a cleanroom used in soft lithography, eliminate the use of silicon wafers, and offer fine control over patterning and cutting/ablation of a hydrogel, thereby increasing reproducibility and eliminating user error that may occur by imprecise alignment of photomasks. Furthermore, the methods of the invention are cell safe, guide tissue development into forming tissues, e.g., anisotropic (aligned) tissues, allow for single cell micropatterning, do not significantly alter surface properties of the hydrogel, e.g., stiffness, and can be used for, e.g., microfluidic technologies including, for example, muscle thin film technologies.

Accordingly, described herein are methods for optical micropatterning of a base, which may be used for producing fluidic devices, and methods of use thereof. The devices produced by the disclosed methods may comprise a solid support structure as a base and a micropatterned hydrogel layer configured to support growth of a functional tissue, e.g., functional muscle tissue. The devices and their method of production are described in further detail below. In some embodiments, the functional muscle tissue comprises cells selected from the group consisting of cardiac muscle cells, ventricular cardiac muscle cells, atrial cardiac muscle cells, striated muscle cells, smooth muscle cells, vascular smooth muscle cells, and combinations thereof.

The devices may be provided with a cell seeding well as part of a kit. Examples of cell seeding wells that may be included in a kit are described and depicted in International Application No. PCT/US2016/045813 (Attorney Docket No.: 117823-10820), the entire contents of which are incorporated herein by reference). The devices may be provided with a growth promoting layer and a plurality of cells disposed on the growth promoting layer.

I. Methods of the Invention

In some embodiments, the methods of the present invention include modifying a surface energy of at least a portion of a surface of a base comprising a cyclic olefin copolymer (COC). Suitable methods to modify a surface energy of at least a portion of a surface of a base comprising a COC include, for example, plasma treatment, and UV/ozone surface treatment. In addition, COC bases are available commercially, and COC pellets are available commercially and can be melted and, using injection molding, formed into any desired shape. The methods of the invention also include forming a hydrogel layer on the surface of the base overlying the portion of the surface having the modified surface energy, the hydrogel layer being susceptible to cross-linking by exposure to light, the hydrogel layer having a surface facing away from the base, wherein the modification of the surface energy of the portion of the surface of the base promotes adhesion of the hydrogel layer to the surface of the base, and exposing at least a portion of the hydrogel layer to UV light in a preselected pattern.

As used herein, the term base refers to a layer or supporting material on which the hydrogel layer is deposited or formed. In some embodiments the base is a rigid material or a semi-rigid material on which the hydrogel is deposited or formed that provides mechanical support for the hydrogel layer (e.g., a substrate).

As used herein, cyclic olefin copolymer (COC) refers to a material (e.g., a base) that is produced by chain copolymerization of cyclic monomers such as 8,9,10-trinorborn-2-ene (norbornene) or 1,2,3,4,4a,5,8,8a-octahydro-1,4:5,8-dimethanonaphthalene (tetracyclododecene) with ethene (such as TOPAS Advanced Polymer's TOPAS, Mitsui Chemical's APEL), or by ring-opening metathesis polymerization of various cyclic monomers followed by hydrogenation (Japan Synthetic Rubber's ARTON, Zeon Chemical's Zeonex and Zeonor). Shin et al. Pure Appl. Chem., Vol. 77, No. 5, pp. 801-814, 2005.

The base including a COC may be advantageous because COCs are chemically resistant to organic solvents, highly biocompatible, easily cut and machined with lasers and a mill, and have low autofluorescence. As described below, a surface energy of the COC over all or a selected area or areas of the base may be modified to enhance or facilitate bonding between the hydrogel layer and the base and a surface energy of the COC base over other selected areas may be modified to inhibit adhesion of cells to the base. For example, a portion or portions of the surface of the base may be modified with an oxygen plasma treatment to enhance for facilitate bonding of part or all of hydrogel layer to the COC base. As another example, laser etching may be employed to modify a surface energy of part of the base to inhibit cell attachment.

In some embodiments, a surface energy of most or all of the surface of the base that will underlie the hydrogel layer is modified relative to a surface energy of the rest of the surface of the base to promote adhesion with or bonding to the micropatterned hydrogel layer. Modifying the surface energy of most or all of the area of the base that will be covered by hydrogel layer to promote boding with the hydrogel layer is suitable for applications in which it is desirable for most or all of the bottom surface of the hydrogel layer to bond to the base (e.g., in embodiments in which a flexible electrode array disposed at least partially between the hydrogel layer and the base is used to measure electrical properties of functional muscle tissue on the hydrogel layer). In other embodiments, the surface energy of the base is modified to promote adhesion with or bonding to the micropatterned hydrogel layer over only a selected portion or portions of the area of the base that will underlie the hydrogel layer. Modifying the surface energy of only a selected portion or portions of the area of the base that will be covered by hydrogel layer to promote bonding with the hydrogel layer is suitable for applications in which it is desirable for a portion or portions of the bottom surface of the hydrogel layer to be unattached to the underlying base (e.g., for muscle tissue strips that have one or more cantilever portions configured to deflect away from the surface of the base in response to contractile forces exerted by muscle tissue on the hydrogel layer). Further description of modification of a surface energy of a portion or portions of a COC base can be found in International Application No. PCT/US2016/045813 (Attorney Docket No.: 117823-10820), the entire contents of which are incorporated herein by reference.

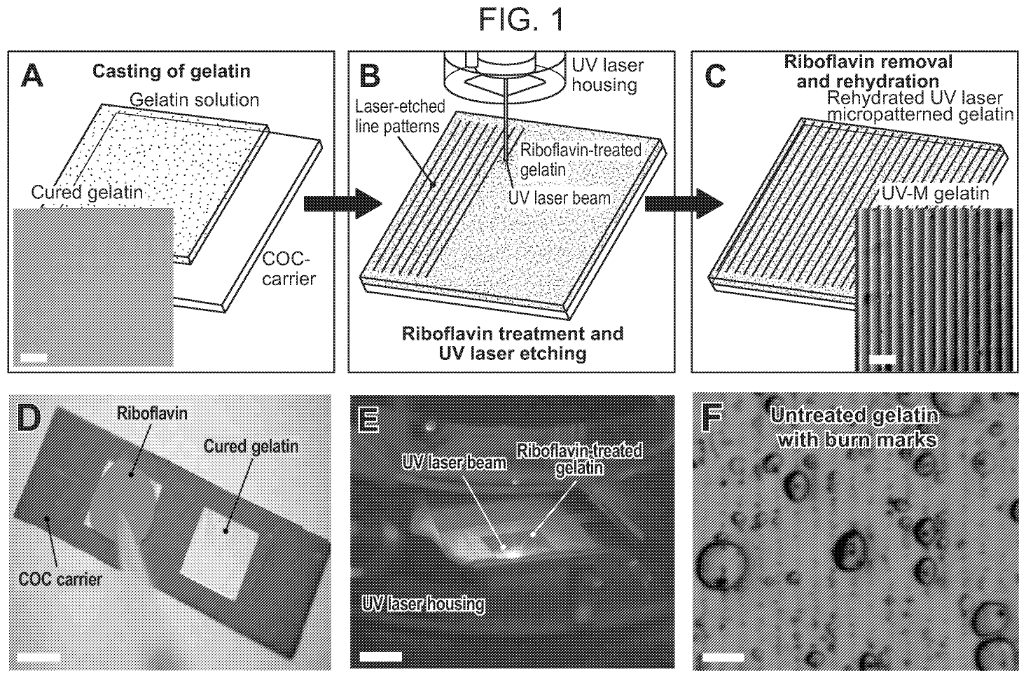

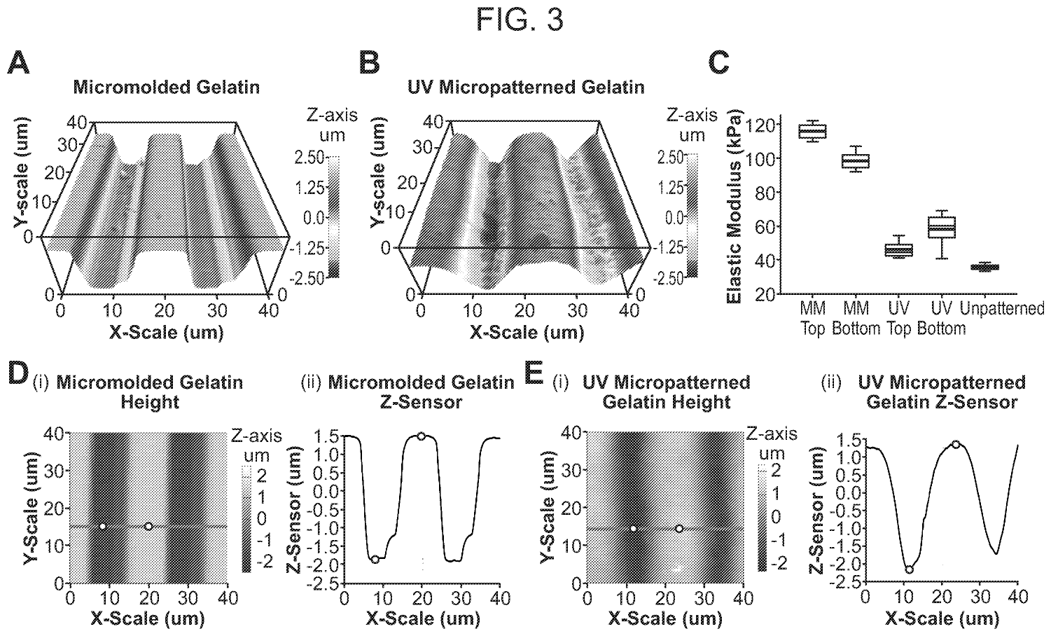

FIGS. 1A-C depict an exemplary method of the invention. In FIG. 1A, a hydrogel (gelatin) cast on a cyclic olefin copolymer (COC) base being treated with a light sensitive solution (e.g., riboflavin 5′ phosphate). In FIG. 1B, the dried crosslinker-treated gelatin COC is patterned using a laser (e.g., a UV microlaser). After hydration, the laser patterned hydrogel has an anisotropic micropatterned surface topography (FIG. 1C), that can be further evaluated using atomic force microscopy (AFM) (see FIG. 3B).

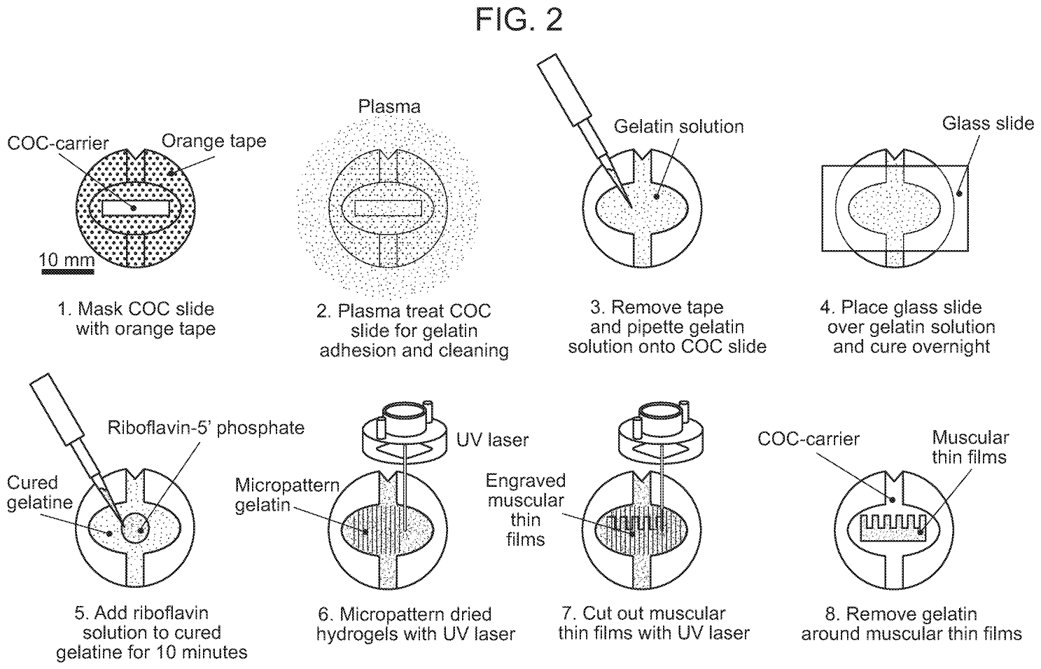

FIG. 2 depicts another exemplary method for optical micropatterning of a hydrogel layer in accordance with one embodiment of the invention.

Suitable hydrogels for use in the methods of the invention include, for example, a gelatin, an alginate, and a poly-acrylic acid (PAA), a UV-linkable hydrogel, including, for example, poly(N-vinylpyrrolidone (PVP), (meth)acrylicated monomers of poly(ethylene glycol), dextran, albumin, (hydroxyethyl)starch, poly-aspartamide, poly(vinyl alcohol), and hyaluronic acid, and mixtures of all of the above. In one embodiment, the hydrogel is a gelatin.

Forming the hydrogel layer, e.g., the gelatin layer, on the surface of the base overlying the portion of the surface having the modified surface energy may include, for example, depositing an aqueous solution comprising a hydrogel on the surface of the base.

The aqueous solution may comprise about 5 to about 20% w/v hydrogel (e.g., gelatin), about 6 to about 20% w/v hydrogel, about 7 to about 20% w/v hydrogel, about 8 to about 20% w/v hydrogel, about 9 to about 20% w/v hydrogel, about 9 to about 19% w/v hydrogel, about 9 to about 18% w/v hydrogel, about 9 to about 17% w/v hydrogel, about 9 to about 16% w/v hydrogel, about 9 to about 15% w/v hydrogel, about 9 to about 14% w/v hydrogel, about 9 to about 13% w/v hydrogel, about 9 to about 12% w/v hydrogel, about 9 to about 11% w/v hydrogel, or about 9 to about 10% w/v hydrogel. In one embodiment, the aqueous solution comprises about 5 to about 20% w/v hydrogel (e.g., gelatin), e.g., about 5, 6, 7, 8, 9, 10, 11, 12, 13, 14, 15, 16, 17, 18, 19, or about 20% w/v hydrogel. In one embodiment, the aqueous solution comprises about 9 to about 10% w/v hydrogel (e.g., gelatin).

In one embodiment, the stiffness of the hydrogel is tuned to mimic the mechanical properties of healthy tissue, such as muscle tissue, e.g., cardiac tissue in vivo, e.g., to have a Young's modulus of about 10, 11, 12, 13, 14, 15, 16, 17, 18, 19, or about 20 kPa. In another embodiment, the stiffness of the hydrogel is tuned to mimic the mechanical properties of diseased tissue, such as muscle tissue, e.g., cardiac tissue in vivo, e.g., to have a Young's modulus of greater than about 45, 46, 47, 48, 49, 50, 51, 52, 53, 54, or about 55 kPa.

The aqueous solution used to deposit the hydrogel on the surface of the base may further comprise additional components. For example, such additional components may include, but are not limited to, a transglutaminase, e.g., a microbial transglutaminase (e.g., when the hydrogel is a gelatin), or Ca⁺² (e.g., when the hydrogel is an alginate), Polycarbodiimide (e.g., when the hydrogel is a PAA), PAA (e.g., when the hydrogel is a PVP). In addition, the aqueous solution may be heated to cross link the hydrogel, e.g., when the hydrogel comprises an ethylacrylate.

Examples of transglutaminases suitable for use in the methods of the invention include, for example, Factor XIII, keratinocyte transglutaminase, tissue transglutaminase, epidermal transglutaminase, prostate transglutaminase, TGM X, TGM Y, and TGM Z.

The concentration of the transglutaminase in the aqueous solution may be at saturation concentration. In some embodiments, the concentration of the transglutaminase is below saturation. In one embodiment, the concentration of the transglutaminase in the aqueous solution is about 4% or more w/v, e.g., about 4.0, about 4.1, about 4.2, about 4.3, about 4.4, about 4.5, about 4.6, about 4.7, about 4.8, about 4.9, about 5.0, about 10, about 15, or about 20% w/v. In another embodiment, the concentration of the transglutaminase is about 4% w/v.

In some embodiments, the hydrogel is a gelatin and the aqueous solution comprises about 9% to about 10% w/v gelatin, e.g., about 9.0, 9.1, 9.2, 9.3, 9.4, 9.5, 9.6, 9.7, 9.8, 9.9, or about 10.0% gelatin, and about 4% w/v microbial transglutaminase. In other embodiments, the hydrogel is a gelatin and the aqueous solution comprises about 4.5% to about 5.5% w/v gelatin, e.g., about 4.5, 4.6, 4.7, 4.8. 4.9, 5.0, 5.1, 5.2, 5.3, 5.4, or about 5.5% gelatin, and about 4% w/v microbial transglutaminase.

Any suitable pre-selected pattern may be used in the methods of the invention. In one embodiment, the preselected pattern is an anisotropic pattern. In another embodiment, the pre-selected pattern is an isotropic pattern. Alternatively, the preselected pattern can be or can include a geometric shape, e.g., a rectangle, a square, a circle, a triangle, etc. In one embodiment, the pattern is a square saw-tooth pattern to produce, e.g., a cantilever, e.g., a cantilevered tissue strip (see, International Application No. PCT/US2016/045813, Attorney Docket No.: 117823-10820), the entire contents of which are incorporated herein by reference.

The pre-selected pattern may include a plurality of lines or a plurality of line segments. In one embodiment, the plurality of lines or a plurality of line segments are substantially parallel. In another embodiment, the pattern comprises a plurality of lines or a plurality of line segments that are substantially parallel and a second plurality of lines or a plurality of line segments that each independently intersect the first plurality of lines or a plurality of line segments at an angle of about 0 to about 90 degrees.

In one embodiment, the plurality of lines or a plurality of line segments have a peak-to-peak line separation in a range of about 0.1 μm to about 1000 μm, from about 1 μm to about 500 μm, from about 1 μm to 250 μm, from about 1 μm to 100 μm, from about 1 μm to 90 μm, from about 1 μm to 80 μm, from about 1 μm to 70 μm, from about 1 μm to 60 μm, from about 1 μm to 50 μm, from about 1 μm to 40 μm, from about 1 μm to 30 μm, from about 1 μm to 20 μm, from about 1 μm to 10 μm, from about 2 μm to 100 μm, from about 2 μm to 90 μm, from about 2 μm to 80 μm, from about 2 μm to 70 μm, from about 2 μm to 60 μm, from about 2 μm to 50 μm, from about 2 μm to 40 μm, from about 2 μm to 30 μm, from about 2 μm to 20 μm, from about 2 μm to 10 μm, from about 1 μm to 100 μm, from about 5 μm to about 100 μm, from about 5 μm to about 90 μm, from about 5 μm to about 80 μm, from about 5 μm to about 70 μm, from about 5 μm to about 60 μm, from about 5 μm to about 50 μm, from about 5 μm to about 40 μm, from about 5 μm to about 30 μm, from about 5 μm to about 20 μm, from about 5 μm to about 20 μm, from about 10 μm to about 100 μm, from about 10 μm to about 90 μm, from about 10 μm to about 80 μm, from about 10 μm to about 70 μm, from about 10 μm to about 60 μm, from about 10 μm to about 50 μm, from about 10 μm to about 40 μm, from about 10 μm to about 30 μm, from about 10 μm to about 20 μm, and from about 10 μm to about 20 μm. In one embodiment, the peak-to-peak line separation is 1 μm to 100 μm, e.g., about 10 μm to about 30 μm. In another embodiment, the peak-to-peak line separation is about 10, about 11, about 12, about 13, about 14, about 15, about 16, about 17, about 18, about 19, about 20, about 25, or about 30 μm. In yet another embodiment, the peak-to-peak line separation is about 15 μm to about 20 μm.

When the pre-selected pattern includes a plurality of lines or a plurality of line segments, the resulting micropattern in the surface of the hydrogel layer may have a peak-to-trough height that falls in a range of about 0.1 μm to about 100 μm, about 0.2 μm to about 100 μm, about 0.3 μm to about 100 μm, about 0.4 μm to about 100 μm, about 0.5 μm to about 100 μm, about 0.5 μm to about 90 μm, about 0.5 μm to about 80 μm, about 0.5 μm to about 70 μm, about 0.5 μm to about 60 μm, about 0.5 μm to about 50 μm, about 0.5 μm to about 40 μm, about 0.5 μm to about 30 μm, about 0.5 μm to about 20 μm, or about 0.5 μm to about 10 μm. In one embodiment, the peak-to-trough height is about 1 μm to about 5 μm. In another embodiment, the peak-to-trough height is about 0.5, 0.6, 0.7, 0.8, 0.9, 1, 2, 3, 4, or about 5 μm. In yet another embodiment, the peak-to-trough height is about 2 μm or about 3 μm.

A laser, such as a microlaser, may be used to expose the portion of the hydrogel layer to light in the pre-selected pattern. The laser may have a beam diameter in a range of about 1 μm to about 100 μm, about 2 μm to about 100 μm, about 5 μm to about 100 μm, about 10 μm to about 100 μm, about 10 μm to about 90 μm, about 10 μm to about 80 μm, about 10 μm to about 70 μm, about 10 μm to about 60 μm, about 10 μm to about 50 μm, about 10 μm to about 40 μm, about 10 μm to about 30 μm, or about 10 μm to about 20 μm. In one embodiment, the laser has a beam diameter in a range of about 10 μm to about 20 μm, e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or about 40 μm. In one embodiment, the beam diameter is about 15 to about 25 μm, e.g., about 20 μm.

Any suitable method may be used to expose the portion of the hydrogel layer to light in the pre-selected pattern. In one embodiment, exposing the portion of the hydrogel layer to light in the preselected pattern comprises serially writing the preselected pattern into the hydrogel layer using a laser, e.g., a microlaser.

The appropriate speed of the laser will depend on the power of the laser which may, in turn, affect the number of repetitions. The number of repetitions can range from 1 to 5 (e.g., 1, 2, 3, 4, or 5 repetitions) in cases of low laser power. In some embodiments, the speed of the laser when serially writing may fall in a range of about 0.0001 W/mm/s (Watts per millimeter/sec) to about 0.005 W/mm/s, about 0.0001 W/mm/s to about 0.004 W/mm/s, about 0.0001 W/mm/s to about 0.003 W/mm/s, about 0.0002 W/mm/s to about 0.003 W/mm/s, about 0.0003 W/mm/s to about 0.003 W/mm/s, about 0.0004 W/mm/s to about 0.003 W/mm/s, about 0.0005 W/mm/s to about 0.003 W/mm/s, about 0.0006 W/mm/s to about 0.003 W/mm/s, about 0.0007 W/mm/s to about 0.003 W/mm/s, about 0.0008 W/mm/s to about 0.003 W/mm/s, about 0.0009 W/mm/s to about 0.003 W/mm/s, about 0.0009 W/mm/s to about 0.002 W/mm/s, or about 0.0009 W/mm/s to about 0.001 W/mm/s. In one embodiment, the speed of the laser is about 0.0009 W/mm/s to about 0.001 W/mm/s.

A variety of different types of light and of different light sources may be used for patterning the hydrogel layer. In certain embodiments, the hydrogel layer is exposed to ultraviolet light. In other embodiments, the hydrogel layer is exposed to visible light. The wavelength of the light can be from about 10 nm to about 600 nm, about 20 nm to about 600 nm, about 50 nm to about 600 nm, about 100 nm to about 600 nm, about 200 nm to about 600 nm, about 300 nm to about 600 nm, about 10 nm to about 500 nm, about 20 nm to about 500 nm, about 50 nm to about 500 nm, about 100 nm to about 500 nm, about 200 nm to about 500 nm, about 300 nm to about 500 nm, about 300 nm to about 450 nm, about 300 nm to about 400 nm, or about 300 nm to about 350 nm. In one embodiment, the wavelength of the light is about 300 nm to about 500 nm. In another embodiment, the wavelength of the light is about 350 nm to about 500 nm. In yet another embodiment, the wavelength of the light is about 500 nm to about 600 nm. In another embodiment, the wavelength of the light is about 350 nm to about 400 nm. In one embodiment, the wavelength of the light is about 530 nm to about 580 nm. In another embodiment, the wavelength of the light is about 350 nm to about 400 nm.

The methods of the invention may further comprise additional steps. For example, in one embodiment, forming the gelatin layer on the surface of the base overlying the portion of the surface having the modified surface energy includes depositing an aqueous solution comprising a hydrogel on the surface of the base overlying the portion of the surface of the base having the modified surface energy. The methods of the invention may further include curing the deposited aqueous solution resulting in a cured layer. In one embodiment, the cured layer may be treated with a second solution that makes the cured layer susceptible to cross-linking by exposure to light and, in some embodiments, the cured layer is rinsed in an aqueous solution, e.g., water, following treatment with the second solution.

Suitable methods and times for curing, e.g., drying, a hydrogel are known to one of ordinary skill in the art. Without being bound by any one particular theory, after about 10 hours, gel strength reaches greater than 95% of final strength and there are no detrimental effects of longer curing times. However, curing times greater than about one month may result in some form of degradation/oxidation that can alter the properties of the hydrogel. Accordingly, the deposited aqueous solution may be cured for at least about 10 hours and up to about one month. For example, the curing time may be about 10, 11, 12, 13, 14, 15, 16, 17, 18, 19, 20, 21, 22, 23, 24, or more hours, but no longer than about one month's time.

Suitable second solutions that may be used to make the cured layer susceptible to cross-linking by exposure to light include aqueous solutions comprising riboflavin-5′ phosphate (e.g., sensitive to light having a wavelength of about 300 nm to about 500 nm), Rose Bengal (e.g., sensitive to light having a wavelength of about 530 nm to about 580 nm), or SU-8 Photoresist (e.g., sensitive to light having a wavelength of about 350-400 nm). In one embodiment, the second solution comprises riboflavin-5′ phosphate.

In certain embodiments, the second solution comprises about 0.01% to about 0.3% w/v riboflavin-5′ phosphate, e.g., about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, or about 0.3% w/v riboflavin-5′ phosphate. In one embodiment, the second solution comprises about 0.05% w/v riboflavin-5′ phosphate.

The methods of the invention may further include hydrating the cured layer in an aqueous solution, e.g., water, prior to treating the cured layer with the second solution. For example, the cured layer may be hydrated for at least about 3 hours for each centimeter of the maximal radius of the hydrogel. For example, a maximal radius of 3 cm of hydrogel requires at least about 9 hours curing time. In one embodiment, the hydrogel is hydrated for at least about 8 hours, e.g., about 8, 9, 10, 11, 12, 13, 14, 15, 16, 17, 18, 19, 20, 21, 22, 23, about 24, or more hours. Alternatively, in lieu of hydrating the cured layer in an aqueous solution, e.g., water, prior to treating the cured layer with the second solution, curing may take place in a humidified chamber having greater than about 90% relative humidity. Subsequently, dependent on the desired application, e.g., regenerative medicine applications, the hydrogel may be removed from the base.

In some embodiments of the invention, e.g., when the methods of the invention include curing and subsequent hydration of the hydrogel, a portion of the surface of the base may be masked using an adhesive mask prior to modifying a surface energy of at least a portion of a surface of a base comprising a COC. In these embodiments, the surface energy of the masked portion of the surface of the base is not modified during the modification of the surface energy of at least a portion of the surface of the base. Subsequently, the adhesive mask may be removed from the surface of the base after hydration of the cured layer.

The methods of the invention may further include cutting through a full thickness of the gelatin layer using a laser after the surface of the gelatin layer has been micropatterned. Additionally, the methods of the invention may further include ablating a portion of the gelatin layer using a laser after the surface of the gelatin layer has been micropatterned. In addition, the methods may further include modifying a surface energy of a portion of the surface of the base surrounding the micropatterned gelatin layer to inhibit cell adhesion to the surface of the base. With respect to the latter, the surface energy of the portion of the surface of the base surrounding the micropatterned gelatin layer may be modified using a laser. Various lasers can be used in these additional steps, as described above. In one embodiment, the laser is a UV laser, e.g., a UV microlaser.

The present invention also provides fluidic devices comprising the optically micropatterned hydrogels of the invention. In one embodiment, the micropatterned surface of the hydrogel is configured to support cell adhesion and tissue growth and the methods of the invention may further include seeding cells, e.g., muscle, lung, pancreas, neural, bone, dental, liver, kidney, smooth muscle, e.g., uterine tissue, vascular smooth muscle, aortic valve tissue, skin, etc., on the micropatterned surface of the hydrogel.

Suitable fluidic devices are described in U.S. Provisional Application No. 62/202,213, filed on Aug. 7, 2015, and International Application No.: PCT/US2016/045813 (Attorney Docket No.: 117823-10820). The entire contents of each of the foregoing applications are incorporated herein by reference.

In some embodiments, the micropatterned surface of the hydrogel is configured to support growth of a functional muscle tissue, e.g., the pre-selected micropattern includes a plurality of lines or a plurality of line segments, and muscle cells are seeded on the micropatterned surface of the hydrogel. The muscle cells may be cardiac muscle cells, ventricular cardiac muscle cells, atrial cardiac muscle cells, striated muscle cells, smooth muscle cells, or vascular smooth muscle cells, or combinations thereof.

As used herein, a “functional muscle tissue” refers to a muscle tissue prepared in vitro which displays at least one physical characteristic typical of the muscle tissue in vivo; and/or at least one functional characteristic typical of the muscle tissue in vivo, i.e., is functionally active.

For example, a physical characteristic of a functional muscle tissue may comprise the presence of parallel (to the long axis of the cells) myofibrils with or without sarcomeres aligned in z-lines, and/or that the myofibrils cross cell-to-cell junctions, and/or that the cells maintain a registered array or sarcomeres, and/or that the cells form cell-to-cell gap junctions and/or cell-to-cell adherens junctions.

Methods to determine such physical characteristics include, for example, microscopic analyses, such as, fluorescent microscopy, confocal microscopy, two-photon microscopy, and the like, immunohistochemical analyses, e.g., staining for connexin 43 to determine if the cells have formed electrically-competent junctions, staining for β-catenin to determine if the cells have formed mechanically-competent junctions, staining for β-actin and determining, e.g., the orientational order parameter (OOP) of the networks to determine if the cells have formed registered myofibrils.

A functional characteristic of a functional muscle tissue may comprise an electrophysiological activity, such as an action potential, or biomechanical activity, such as contraction. For example, the cells of a functional muscle tissue may be mechanically and electrically integrated, e.g., the cells synchronously contract, and/or the cells generate a contractile force, and/or the contractions of the cells are in phase, and/or the contractile force at the medial cell-to-cell junctions of the cells are about the same, and/or the cells exhibit synchronous Ca²⁺ transients, and/or the cells exhibit substantially the same Ca²⁺ levels, and/or the cells exhibit peak systolic and/or diastolic forces that are about the same.

Methods to determine such functional characteristics include, for example, microscopic analyses, such as fluorescent microscopy, confocal microscopy, two-photon microscopy, and the like, immunohistochemical analyses, e.g., vinculin staining, traction force microscopy, ratiometric Ca²⁺ imaging, and optical mapping of the action potentials.

To prepare a functional muscle tissue, a micropatterned surface of a hydrogel prepared as described herein is placed in culture with a myocyte suspension, the cells are allowed to settle and adhere to the micropatterned hydrogel layer. In the case of an adhesive surface treatment, cells bind to the micropatterned surface of the hydrogel in a manner dictated by the micro-scale topological features on the micropatterned surface of the hydrogel and the cells respond to the features in terms of maturation, growth and function. The cells on the hydrogel may be cultured, e.g., in an incubator, under physiologic conditions (e.g., at 37° C.) until the cells form a two-dimensional (2D) tissue (i.e., a layer of cells that is less than about 200 microns thick, or, in particular embodiments, less than about 100 microns thick, less than about 50 microns thick, or even just a monolayer of cells), the anisotropy or isotropy of which is determined by the micropatterned topological features.

Any appropriate cell culture method may be used to establish the tissue on the micropatterned surface of the hydrogel. The seeding density of the cells will vary depending on the cell size and cell type, but can easily be determined by methods known in the art. In some embodiments, myocytes are cultured in the presence of, e.g., a fluorophore, nanoparticles and/or fluorescent beads, e.g., fluorospheres. In one embodiment, a fluorophore, a nanoparticle and/or a fluorescent bead, e.g., a fluorosphere, is mixed with the hydrogel.

The cells, e.g., the myocytes, may be normal cells, abnormal cells (e.g., those derived from a diseased tissue, or those that are physically or genetically altered to achieve an abnormal or pathological phenotype or function), normal or diseased cells derived from embryonic stem cells or induced pluripotent stem cells, or cells comprising a genetic construct, such as an expression vector expressing an optogenetic gene, e.g., a light sensitive ion channel (e.g., channelrhodopsin (ChR2), C1V1, Chrimson, Chronos, SSFO, ArchT, ChETA, NpHR, SwiChR, iC1C2, or the like). Cells, e.g., mycocytes can be cultured in vitro, derived from a natural source, genetically engineered, or produced by any other means. Any natural source of myocytes may be used, including from neonates, e.g., mouse and rat neonates.

Suitable myocytes for the preparation of a functional muscle tissue include, cardiomyocytes, such as ventricular or atrial cardiac cells vascular smooth muscle cells, airway smooth muscle cells, and striated muscle cells, such as skeletal muscle cells, and combinations thereof.

The term “stem cell” as used herein, refers to an undifferentiated cell that is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation”.

The term “embryonic stem cell” is used to refer to the pluripotent stem cells of the inner cell mass of the embryonic blastocyst (see, e.g., U.S. Pat. Nos. 5,843,780, 6,200,806, the entire contents of each of which are incorporated herein by reference). Such cells can similarly be obtained from the inner cell mass of blastocysts derived from somatic cell nuclear transfer (see, for example, U.S. Pat. Nos. 5,945,577, 5,994,619, 6,235,970, the entire contents of each of which are incorporated herein by reference). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells.

The term “progenitor cell” is used herein to refer to cells that have a cellular phenotype that is more primitive (e.g., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate. Furthermore, the term “progenitor cell” is used herein synonymously with “stem cell.”

In one embodiment, progenitor cells suitable for use in the claimed devices and methods are Committed Ventricular Progenitor (CVP) cells as described in PCT Application No. WO 2010/042856, entitled “Tissue Engineered Mycocardium and Methods of Productions and Uses Thereof,” filed Oct. 9, 2009, the entire contents of which are incorporated herein by reference.

Suitable stem cells for use in the present invention include stem cells that will differentiate into a myocyte, the differentiated progeny of such stem cells, and the dedifferentiated progeny of myocytes, and include embryonic (primary and cell lines), fetal (primary and cell lines), adult (primary and cell lines) and iPS (induced pluripotent stem cells). The stem cells may be normal stem cells, abnormal stem cells (e.g., those derived from a diseased tissue, or those that are physically or genetically altered to achieve an abnormal or pathological phenotype or function), normal or diseased cells derived from embryonic stem cells or induced pluripotent stem cells, or cells comprising a genetic construct, such as an expression vector expressing an optogenetic gene, e.g., a light sensitive ion channel (e.g., channelrhodopsin (ChR2), C1V1, Chrimson, Chronos, SSFO, ArchT, ChETA, NpHR, SwiChR, iC1C2, or the like).

Stem cells can be cultured in vitro, derived from a natural source, genetically engineered, or produced by any other means. Any natural source of cells may be used. For example, in one embodiment the stem cells suitable for use in the present invention, e.g., stem cells that give rise to myocytes, may be selected from the group consisting of a primary embryonic stem cell, a stem cell from an embryonic stem cell line, a primary fetal stem cell, a stem cell from a fetal stem cell line, a primary adult stem cell, a stem cell from an adult stem cell line, a stem cell de-differentiated from an adult cell, and an induced pluripotent stem cell (iPS).

II. Methods of Use of the Devices of the Invention

The micropatterned hydrogels of the present invention are useful for, among other things, measuring cell activities or functions, e.g., muscle cell activities and functions, investigating cell developmental biology and disease pathology, e.g., muscle cell developmental biology and disease pathology, drug delivery use in tissue engineering, e.g., cell scaffolding, regenerative medicine and wound healing, as well as in drug discovery and toxicity testing.

Other uses of the exemplary micropatterned hydrogels of the invention include, but are not limited to, manufacture of engineered tissue and organs, including structures such as patches or plugs of tissues or matrix material, prosthetics, and other implants, tissue scaffolding for, e.g., fractal neural and/or vascular networks, repair or dressing of wounds, hemostatic devices, devices for use in tissue repair and support such as sutures, surgical and orthopedic screws, and surgical and orthopedic plates, natural coatings or components for synthetic implants, cosmetic implants and supports, repair or structural support for organs or tissues, substance delivery, bioengineering platforms, platforms for testing the effect of substances upon cells, cell culture, cell scaffolding, drug delivery, wound healing, food products, enzyme immobilization, forming a food item, forming a medicinal item, forming a cosmetic item, forming a structure inside a body cavity, and the like and numerous other uses.

Tissue scaffolds and structures prepared using the hydrogels of the invention are good candidates for tissue engineering due to their high surface to mass ratio, high porosity for, e.g., breathability, encapsulation of active substances, and because the structures can be easily molded into different shapes.

Tissue engineering applications for structures made using the hydrgels of the invention include, but are not limited to orthopedic, muscular, vascular and neural prostheses, and regenerative medicine. Madurantakam, et al. (2009) Nanomedicine 4:193-206; Madurantakam, P. A., et al. (2009) Biomaterials 30(29):5456-5464; Xie, et al. (2008) Macromolecular Rapid Communications 29:1775-1792.

As hydrogels, such as alginate and gelatin are edible and approved for human consumption in the United States and Europe, the micropatterned hydrogels produced according to the methods disclosed herein may also be used to generate food products

The fluidic devices comprising the optically micropatterned hydrogels of the invention of the invention configured to support cell adhesion and tissue growth, e.g., muscle, lung, pancreas, neural, bone, dental, liver, kidney, etc. tissue on the micropatterned surface of the hydrogel may be used to evaluate numerous physiologically relevant cell parameters, such as muscle cell parameters, e.g., muscle activities, e.g., biomechanical and electrophysiological activities. For example, in one embodiment, the devices of the present invention can be used in contractility assays for contractile cells, such as muscular cells or tissues, such as chemically and/or electrically stimulated contraction of vascular, airway or gut smooth muscle, cardiac muscle, vascular endothelial tissue, or skeletal muscle. In addition, the differential contractility of different muscle cell types to the same stimulus (e.g., pharmacological and/or electrical) can be studied.

In another embodiment, the devices of the present invention can be used for measurements of solid stress due to osmotic swelling of cells. For example, as the cells swell the tissue, e.g., muscle tissue, will contract/bend and as a result, volume changes, force and points of rupture due to cell swelling can be measured.

In another embodiment, the devices of the present invention can be used for pre-stress or residual stress measurements in cells. For example, vascular smooth muscle cell remodeling due to long-term contraction in the presence of endothelin-1 can be studied.

Further still, the devices of the present invention can be used to study the loss of rigidity in tissue structure after traumatic injury, e.g., traumatic brain injury. Traumatic stress can be applied to vascular smooth muscle thin films as a model of vasospasm. These devices can be used to determine what forces are necessary to cause vascular smooth muscle to enter a hyper-contracted state. These devices can also be used to test drugs suitable for minimizing vasospasm response or improving post-injury response and returning vascular smooth muscle contractility to normal levels more rapidly.

In other embodiments, the devices of the present invention can be used to study biomechanical responses to paracrine released factors (e.g., vascular smooth muscle dilation due to release of nitric oxide from vascular endothelial cells, or cardiac myocyte dilation due to release of nitric oxide).

In still other embodiments, the devices of the present invention can be used to measure the influence of biomaterials on a biomechanical response. For example, differential contraction of vascular smooth muscle remodeling due to variation in material properties (e.g., stiffness, surface topography, surface chemistry or geometric patterning) of, e.g., a gelatin layer, can be studied.

In further embodiments, the devices of the present invention can be used to study functional differentiation of stem cells (e.g., pluripotent stem cells, multipotent stem cells, induced pluripotent stem cells, and progenitor cells of embryonic, fetal, neonatal, juvenile and adult origin) into contractile phenotypes. For example, undifferentiated cells, e.g., stem cells, are seeded on the devices of the invention and differentiation into a contractile phenotype is observed by observing contraction/bending. Differentiation into an anisotropic tissue may also be observed by quantifying the degree of alignment of sarcomeres and/or quantifying the orientational order parameter (OOP). Differentiation can be observed as a function of: co-culture (e.g., co-culture with differentiated cells), paracrine signaling, pharmacology, electrical stimulation, magnetic stimulation, thermal fluctuation, transfection with specific genes, chemical and/or biomechanical perturbation (e.g., cyclic and/or static strains).

In one embodiment a biomechanical perturbation is stretching of, e.g., the hydrogel during tissue formation. In one embodiment, the stretching is cyclic stretching. In another embodiment, the stretching is sustained stretching.

The devices of the invention are also useful for evaluating the effects of particular delivery vehicles for therapeutic agents e.g., to compare the effects of the same agent administered via different delivery systems, or simply to assess whether a delivery vehicle itself (e.g., a viral vector or a liposome) is capable of affecting the biological activity of the muscle tissue. These delivery vehicles may be of any form, from conventional pharmaceutical formulations, to gene delivery vehicles. For example, the devices of the invention may be used to compare the therapeutic effect of the same agent administered by two or more different delivery systems (e.g., a depot formulation and a controlled release formulation). The devices and methods of the invention may also be used to investigate whether a particular vehicle may have effects of itself on the tissue. As the use of gene-based therapeutics increases, the safety issues associated with the various possible delivery systems become increasingly important. Thus, the devices of the present invention may be used to investigate the properties of delivery systems for nucleic acid therapeutics, such as naked DNA or RNA, viral vectors (e.g., retroviral or adenoviral vectors), liposomes and the like. Thus, the test compound may be a delivery vehicle of any appropriate type with or without any associated therapeutic agent.

In other embodiments, the devices of the invention can be used to evaluate the effects of a test compound on a contractile function of a functional muscle tissue. Accordingly, in one aspect, the present invention provides methods for identifying a compound that modulates a contractile function of a functional muscle tissue. The methods include providing any one of the devices disclosed herein comprising a functional muscle tissue, e.g., a functional muscle tissue comprising a substantially confluent layer of muscle cells and/or a functional muscle tissue strip, contacting the functional muscle tissue with a test compound; and determining the effect of the test compound on a contractile function in the presence and absence of the test compound, wherein a modulation of the contractile function in the presence of the test compound as compared to the contractile function in the absence of the test compound indicates that the test compound modulates a contractile function, thereby identifying a compound that modulates a contractile function.

In one embodiment, the contractile function is a biomechanical activity, e.g., contractility, cell stress, cell swelling, and/or rigidity. In some embodiment, fluorescent beads are included in the gelatin layer and the biomechanical activity is determined using traction force microscopy.

In some embodiments, e.g., when the device include a functional muscle tissue strip or a plurality of functional muscle tissue strips, determining a biomechanical activity includes determining the degree of contraction, i.e., the degree of curvature or bend of the tissue strip, and the rate or frequency of contraction/rate of relaxation compared to a normal control or control strip in the absence of the test compound (see, e.g., U.S. Pat. No. 9,012,172 and U.S. Patent Publication No. 20140342394, the entire contents of each of which are incorporated herein by reference).

In another embodiment, the contractile function is an electrophysiological activity, e.g., an electrophysiological profile comprising a voltage parameter selected from the group consisting of action potential, action potential morphology, action potential duration (APD), conduction velocity (CV), refractory period, wavelength, restitution, bradycardia, tachycardia, reentrant arrhythmia, and/or a calcium flux parameter, e.g., intracellular calcium transient, transient amplitude, rise time (contraction), decay time (relaxation), total area under the transient (force), restitution, focal and spontaneous calcium release, and wave propagation velocity. For example, a decrease in a voltage or calcium flux parameter of a muscle tissue comprising cardiomyocytes upon contraction of the tissue in the presence of a test compound would be an indication that the test compound is cardiotoxic.

In yet another embodiment, the devices of the present invention can be used in pharmacological assays for measuring the effect of a test compound on the stress state of a tissue. For example, the assays may involve determining the effect of a drug on tissue stress and structural remodeling of the muscle tissue. In addition, the assays may involve determining the effect of a drug on cytoskeletal structure (e.g., sarcomere alignment) and, thus, the contractility of the muscle tissue.

In another embodiment, the devices of the invention may be used to determine the toxicity of a test compound by evaluating, e.g., the effect of the compound on an electrophysiological response of a muscle tissue. For example, opening of calcium channels results in influx of calcium ions into the cell, which plays an important role in excitation-contraction coupling in cardiac and skeletal muscle fibers. The reversal potential for calcium is positive, so calcium current is almost always inward, resulting in an action potential plateau in many excitable cells. These channels are the target of therapeutic intervention, e.g., calcium channel blocker sub-type of anti-hypertensive drugs. Candidate drugs may be tested in the electrophysiological characterization assays described herein to identify those compounds that may potentially cause adverse clinical effects, e.g., unacceptable changes in cardiac excitation, that may lead to arrhythmia.

For example, unacceptable changes in cardiac excitation that may lead to arrhythmia include, e.g., blockage of ion channel requisite for normal action potential conduction, e.g., a drug that blocks Na⁺ channel would block the action potential and no upstroke would be visible; a drug that blocks Ca²⁺ channels would prolong repolarization and increase the refractory period; blockage of K⁺ channels would block rapid repolarization, and, thus, would be dominated by slower Ca²⁺ channel mediated repolarization.

In addition, metabolic changes may be assessed to determine whether a test compound is toxic by determining, e.g., whether contacting with a test compound results in a decrease in metabolic activity and/or cell death. For example, detection of metabolic changes may be measured using a variety of detectable label systems such as fluormetric/chrmogenic detection or detection of bioluminescence using, e.g., AlamarBlue fluorescent/chromogenic determination of REDOX activity (Invitrogen), REDOX indicator changes from oxidized (non-fluorescent, blue) state to reduced state(fluorescent, red) in metabolically active cells; Vybrant MTT chromogenic determination of metabolic activity (Invitrogen), water soluble MTT reduced to insoluble formazan in metabolically active cells; and Cyquant NF fluorescent measurement of cellular DNA content (Invitrogen), fluorescent DNA dye enters cell with assistance from permeation agent and binds nuclear chromatin. For bioluminescent assays, the following exemplary reagents may be used: Cell-Titer Glo luciferase-based ATP measurement (Promega), a thermally stable firefly luciferase glows in the presence of soluble ATP released from metabolically active cells.

In another aspect, the present invention provides methods for identifying a compound useful for treating or preventing a muscle disease. The methods include providing any one of the devices disclosed herein comprising a functional muscle tissue, e.g., a functional muscle tissue comprising a substantially confluent layer of muscle cells and/or a functional muscle tissue strip; contacting a plurality of the muscle tissues with a test compound; and determining the effect of the test compound on a contractile function in the presence and absence of the test compound, wherein a modulation of the contractile function in the presence of the test compound as compared to the contractile function in the absence of the test compound indicates that the test compound modulates a contractile function, thereby identifying a compound useful for treating or preventing a muscle disease. For example, by determining a biomechanical activity of the functional muscle tissue in the presence and absence of a test compound, an increase in the degree of contraction or rate of contraction indicates, e.g., that the compound is useful in treatment or amelioration of pathologies associated with myopathies such as muscle weakness or muscular wasting. Such a profile also indicates that the test compound is useful as a vasocontractor. A decrease in the degree of contraction or rate of contraction is an indication that the compound is useful as a vasodilator and as a therapeutic agent for muscle or neuromuscular disorders characterized by excessive contraction or muscle thickening that impairs contractile function.

Compounds evaluated in this manner are useful in treatment or amelioration of the symptoms of muscular and neuromuscular pathologies such as those described below. Muscular Dystrophies include Duchenne Muscular Dystrophy (DMD) (also known as Pseudohypertrophic), Becker Muscular Dystrophy (BMD), Emery-Dreifuss Muscular Dystrophy (EDMD), Limb-Girdle Muscular Dystrophy (LGMD), Facioscapulohumeral Muscular Dystrophy (FSH or FSHD) (Also known as Landouzy-Dejerine), Myotonic Dystrophy (MMD) (Also known as Steinert's Disease), Oculopharyngeal Muscular Dystrophy (OPMD), Distal Muscular Dystrophy (DD), and Congenital Muscular Dystrophy (CMD). Motor Neuron Diseases include Amyotrophic Lateral Sclerosis (ALS) (Also known as Lou Gehrig's Disease), Infantile Progressive Spinal Muscular Atrophy (SMA, SMA1 or WH) (also known as SMA Type 1, Werdnig-Hoffman), Intermediate Spinal Muscular Atrophy (SMA or SMA2) (also known as SMA Type 2), Juvenile Spinal Muscular Atrophy (SMA, SMA3 or KW) (also known as SMA Type 3, Kugelberg-Welander), Spinal Bulbar Muscular Atrophy (SBMA) (also known as Kennedy's Disease and X-Linked SBMA), Adult Spinal Muscular Atrophy (SMA) Inflammatory Myopathies include Dermatomyositis (PM/DM), Polymyositis (PM/DM), Inclusion Body Myositis (IBM). Neuromuscular junction pathologies include Myasthenia Gravis (MG), Lambert-Eaton Syndrome (LES), and Congenital Myasthenic Syndrome (CMS). Myopathies due to endocrine abnormalities include Hyperthyroid Myopathy (HYPTM), and Hypothyroid Myopathy (HYPOTM). Diseases of peripheral nerves include Charcot-Marie-Tooth Disease (CMT) (Also known as Hereditary Motor and Sensory Neuropathy (HMSN) or Peroneal Muscular Atrophy (PMA)), Dejerine-Sottas Disease (DS) (Also known as CMT Type 3 or Progressive Hypertrophic Interstitial Neuropathy), and Friedreich's Ataxia (FA). Other Myopathies include Myotonia Congenita (MC) (Two forms: Thomsen's and Becker's Disease), Paramyotonia Congenita (PC), Central Core Disease (CCD), Nemaline Myopathy (NM), Myotubular Myopathy (MTM or MM), Periodic Paralysis (PP) (Two forms: Hypokalemic—HYPOP—and Hyperkalemic—HYPP) as well as myopathies associated with HIV/AIDS.

The methods and devices of the present invention are also useful for identifying therapeutic agents suitable for treating or ameliorating the symptoms of metabolic muscle disorders such as Phosphorylase Deficiency (MPD or PYGM) (Also known as McArdle's Disease), Acid Maltase Deficiency (AMD) (Also known as Pompe's Disease), Phosphofructokinase Deficiency (PFKM) (Also known as Tarui's Disease), Debrancher Enzyme Deficiency (DBD) (Also known as Cori's or Forbes' Disease), Mitochondrial Myopathy (MITO), Carnitine Deficiency (CD), Carnitine Palmityl Transferase Deficiency (CPT), Phosphoglycerate Kinase Deficiency (PGK), Phosphoglycerate Mutase Deficiency (PGAM or PGAMM), Lactate Dehydrogenase Deficiency (LDHA), and Myoadenylate Deaminase Deficiency (MAD).

In addition to the disorders listed above, the screening methods described herein are useful for identifying agents suitable for reducing vasospasms, heart arrhythmias, and cardiomyopathies.

Vasodilators identified as described above are used to reduce hypertension and compromised muscular function associated with atherosclerotic plaques. Smooth muscle cells associated with atherosclerotic plaques are characterized by an altered cell shape and aberrant contractile function. Such cells are used to prepare a functional muscle tissue on a device of the invention, exposed to candidate compounds as described above, and a contractile function evaluated as described above. Those agents that improve cell shape and function are useful for treating or reducing the symptoms of such disorders.

Smooth muscle cells and/or striated muscle cells line a number of lumen structures in the body, such as uterine tissues, airways, gastrointestinal tissues (e.g., esophagus, intestines) and urinary tissues, e.g., bladder. The function of smooth muscle cells on thin films in the presence and absence of a candidate compound may be evaluated as described above to identify agents that increase or decrease the degree or rate of muscle contraction to treat or reduce the symptoms associated with a pathological degree or rate of contraction. For example, such agents are used to treat gastrointestinal motility disorders, e.g., irritable bowel syndrome, esophageal spasms, achalasia, Hirschsprung's disease, or chronic intestinal pseudo-obstruction.

Any of the screening methods of the invention generally comprise determining the effect of a test compound on a functional muscle tissue as a whole, however, the methods of the invention may comprise further evaluating the effect of a test compound on an individual cell type(s) of the muscle tissue.

As used herein, the various forms of the term “modulate” are intended to include stimulation (e.g., increasing or upregulating a particular response or activity) and inhibition (e.g., decreasing or downregulating a particular response or activity).

As used herein, the term “contacting” (e.g., contacting a functional muscle tissue with a test compound) is intended to include any form of interaction (e.g., direct or indirect interaction) of a test compound and a functional muscle tissue. The term contacting includes incubating a compound and a functional muscle tissue together (e.g., adding the test compound to a functional muscle tissue in culture).

Test compounds, may be any agents including chemical agents (such as toxins), small molecules, pharmaceuticals, peptides, proteins (such as antibodies, cytokines, enzymes, and the like), nanoparticles, and nucleic acids, including gene medicines and introduced genes, which may encode therapeutic agents, such as proteins, antisense agents (i.e., nucleic acids comprising a sequence complementary to a target RNA expressed in a target cell type, such as RNAi or siRNA), ribozymes, and the like.

The test compound may be added to a tissue by any suitable means. For example, the test compound may be added drop-wise onto the surface of a device of the invention and allowed to diffuse into or otherwise enter the device, or it can be added to the nutrient medium and allowed to diffuse through the medium. In one embodiment, the screening platform includes a microfluidics handling system to deliver a test compound and simulate exposure of the microvasculature to drug delivery. In one embodiment, a solution comprising the test compound may also comprise fluorescent particles, and a muscle cell function may be monitored using Particle Image Velocimetry (PIV).

In certain embodiments, the methods of the invention are high throughput methods, where a plurality of test compositions or conditions is screened. For example, in certain embodiments, a library of compositions is screened, where each composition of the library is individually contacted to the co-cultures in order to identify which agents suitable for use as described herein.

In one aspect, any of the methods of the invention may further include applying a stimulus, such as an electrical stimulus or a chemical stimulus, or, in the case of cells expressing an optogenetic gene, a light stimulus, to the cells. In one embodiment, the cells are simulated with an alternating current of 10 μA.

The practice of the presently disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements or method steps, those elements or steps may be replaced with a single element or step. Likewise, a single element or step may be replaced with a plurality of elements or steps that serve the same purpose. Further, where parameters for various properties are specified herein for exemplary embodiments, those parameters may be adjusted up or down by 1/20th, 1/10th, ⅕th, ⅓rd, ½, etc., or by rounded-off approximations thereof, unless otherwise specified. Moreover, while exemplary embodiments have been shown and described with references to particular embodiments thereof, those of ordinary skill in the art will understand that various substitutions and alterations in form and details may be made therein without departing from the scope of the invention. Further still, other aspects, functions and advantages are also within the scope of the invention.

This invention is further illustrated by the following examples which should not be construed as limiting. The entire contents of all references, patents and published patent applications cited throughout this application, as well as the Figures, are hereby incorporated herein by reference.

### Example 1: Methods for Micropatterning Hydrogel Layers

FIG. 2 is a schematic showing an exemplary method for producing a micropatterned hydrogel by optical patterning in accordance with one embodiment of the invention. The steps shown therein are as follows:
1. A tape masked COC slide was plasma treated to activate and clean the exposed polymer surface.
2. An aqueous solution with 10% w/v gelatin and 4% w/v microbial transglutaminase was deposited onto the plasma treated surface.
3. Gelatin was cast with a glass slide and cured for 12 hours.
4. After 12 hours, the gelatin was hydrated in water to remove the glass slide and tape.
5. The tape was peeled from the COC slide.
6. The gelatin was treated with a riboflavin 5′ phosphate solution for 10 minutes, then rinsed in water.
7. The gelatin was dried.
8. The gelatin was patterned with a 355 wavelength UV laser (LPKF Protolaser U3).
9. The patterned gelatin was rinsed thoroughly with water, e.g., prior to cell seeding.

### Hydrogel Fabrication

COC slides (specifically, TOPAS COC slides produced by Topas Advanced Polymers) for cell culture (75 mm×25 mm×0.27 mm, Polylinks, Arden, N.C.) were covered with low adhesive tape (3M, St. Paul, Minn.). The tape was scored with the LPKF UV laser engraving system (LPKF Laser and Electronics, Tualatin, Oreg.) into mm squares or mm ellipses and rectangles. The tape was peeled away so that the squares and inner rectangles within the ellipses were exposed for plasma treatment. COC slides were plasma treated for 5 minutes using a PLASMA PPREP III reactor (Structure Probe, Inc. West Chester, Pa.). To make the gelatin solution, 20% w/v gelatin (Sigma-Aldrich, St. Louis, Mo.) was dissolved at 65° C. Cross-linking agent, 8% microbial transglutaminase (mTG) (Ajinomoto, Fort Lee, N.J.) solution, was warmed to 37° C., degassed in a vacuum, and heated back to 37° C. until fully dissolved. Equal parts gelatin and mTG were mixed, resulting in a final concentration of 10% w/v and 4% w/v, respectively. Tape was peeled as necessary and the gelatin solution was pipetted onto the COC slides. A glass microscope slide cleaned with 70% ethanol was gently pressed against the gelatin droplet and cured overnight. Micromolded gelatin hydrogels were fabricated as previously described (McCain et al. Biomaterials, 2014; 35(21):5462-71). The next day, the gelatin was re-hydrated with water and the glass slide was carefully peeled off the gelatin. Hydrated gelatin was treated with 0.05% riboflavin-5′phosphate (Sigma Aldrich) for 10 minutes. COC slides were washed with water for 10 minutes and dried under filtered air for 30 minutes. Once samples were completely dry, hydrogels were patterned and muscle strips were cut out by the UV laser. All samples were washed overnight in phosphate buffer solution (Thermofisher Scientific, Waltham, Mass.). All solutions described were made up in ULTRAPURE DNAse/RNAse free distilled water (Thermofisher Scientific).

### UV Laser Patterning

Designs for cell patterning were created using CORELDRAW graphic design software (Corel Inc., Ottawa, Canada) and exported into CIRCUITCAM and LPKF CIRCUITMASTER computer aided manufacturing software (produced by DCT Co., Ltd in Tianjin, China and LPKF Laser & Electronics AG, respectively). Prior to laser cutting, a micrometer was used to measure gelatin and COC thickness to improve laser focus. Lines were engraved into hydrogels (15 μm×7 μm spacing) and muscle strip cantilevers (2 mm×1.3 mm) were cut with the PROTOLASER U3 laser engraver (produced by LPKF Laser & Electronics AG). The following laser settings produced patterning lines: power=0.13 W, frequency=50 kHz, mark speed=[80-160 mm/s], 1 repetition. The following laser settings were used to cut through gels to produce muscle strip cantilevers: power=0.13 W, frequency=50 kHz, mark speed=50 mm/s, 30-50 repetitions. Mark speed and repetitions for cutting cantilevers were altered according to gelatin thickness and verified using stereoscope microscopy. Remaining gelatin was peeled away from the muscle strip cantilevers. Cantilevers were lifted off the COC to loosen the gelatin from the plastic. Patterns and muscle strips were imaged using a Leica Stereomicroscope and Nikon camera.

### Atomic Force Microscopy Imaging

Fluidic Atomic Force Microscopy (AFM) imaging was performed using the MFP-3D AFM system with an open fluid droplet containing de-ionized water (Asylum Research, Santa Barbara, Calif.). All topography images for hydrated hydrogels were collected in contact mode with soft, gold coated silicon nitride bio-levers with an estimated contact force of 1-10 nN (Olympus TR400PB, Asylum Research Probe Store, Santa Barbara, Calif.). After collecting a contact mode image of each gel sample in DI water, the tip was placed on three independent sites for the top and bottom of the pattern in order to collect at least twenty five Force Distance Curves (FDCs) from each site. The FDCs were analyzed using the JKR model to estimate the elastic modulus of the samples.

### Cell Culture

Neonatal rat ventricular myocytes were isolated from two day old neonatal Sprague-Dawley rats according to protocols approved by the Harvard University Animal Care and Use Committee. After extraction, ventricles were homogenized in Hanks balanced salt solution followed by overnight trypsinization and digestion with collagenase at 4° C. (1 mg/mL, Worthington Biochemical Corp., Lakewood, N.J.). Cell solutions were strained and re-suspended in M199 culture media supplemented with 10% heat-inactivated fetal bovine serum, 10 mM HEPES, 0.1 mM MEM nonessential amino acids, 20 mM glucose, 2 mM L-glutamine, 1.5 mM vitamin B-12, and 50 U/mL penicillin, and pre-plated twice to reduce non-myocyte cell populations. Cardiac myocytes in a density of 2500 cells/mm² were seeded for each well of a 8-well dish. Media was exchanged to maintenance media containing 2% fetal bovine serum (FBS) every 48 hours. Human iPS-derived cardiomyocytes (hiPSCs) (Axiogenesis, Cologne, Germany) were thawed from vials 2 days prior to cell seeding onto cell patterns in Cor.4U medium according to manufacturer's protocols. Cells were typsinized after 2 days in culture with 0.25% trypsin-EDTA (ThermoFisher Scientific) for 5 minutes and washed with Cor.4U medium. Medium was collected into 15 mL conical tubes and centrifuged at 200×g for 5 minutes. Medium was aspirated to leave a pellet of hiPSCs and resuspended with 500 ul of Cor.4U medium. Cells were counted and dispersed on to line patterns at a seeding density of 2500 cells/mm2. Human iPS-derived cardiomyocytes were cultured for 9 days with media changes every 48 hours prior to fixation.

### Immunostaining and OOP Analysis

Engineered cardiac tissues were pre-fixed with 2% paraformaldehyde (Electron Microscopy Sciences, Hatfield, Pa.) for 2 minutes, then fixed with fresh 4% paraformaldehyde and 0.5% Triton-X (Sigma-Aldrich, St. Louis, Mo.) for 8 minutes. Tissues were incubated with 5% BSA for 30 minutes followed by incubation with primary antibodies against sarcomeric α-actinin (Sigma-Aldrich, St. Louis, Mo.), DAPI (Invitrogen, Carlsbad, Calif.) and Alexa Fluor 546 phalloidin for 60 minutes at room temperature. Following washes with 0.5% BSA in phosphate buffer solution, secondary antibodies against mouse IgG conjugated to Alexa Fluor 488 (Invitrogen, Carlsbad, Calif.) were incubated on tissues for 60 minutes. For each coverslip 8-10 fields of view were imaged using a Leica SP5 X MP inverted confocal microscope with 63×/1.3 glycerol objective (Wetzler, Germany). For each coverslip, the fields of view images were stitched up into one mosaic image, the overall orientation angles of α-actinin was calculated as previously described (Feinberg et al., Biomaterials, 2012). Custom MATLAB software (MathWorks, Natick, Mass.) was used to calculate Orientational Order Parameter (OOP) for each mosaic image. The OOPs for each condition were averaged and statistically compared by student's t-test.

### Muscle Tissue Strip Experiments and Analysis

Muscle tissue strip cantilever experiments were performed as previously described (McCain et al. Biomaterials, 2014;35(21):5462-71). Tissues were paced from 0 to 2 Hz using a MyoPacer Cell Stimulator (IonOptix, Milton, Mass.). Movies were imported into ImageJ Fiji image processing software to measure cantilever displacement using ImageJ line tool. The radius of curvature for each cantilever was calculated using the x-projection and original length (Grosberg et al. Lab Chip, 2011). The radius of curvature, thickness, and elastic modulus of each cantilever was used to calculate stress using modified Stoney's equation (Feinberg et al, Science, 2007, 317(5843):1366-70). The average elastic modulus calculated from atomic force microscopy was used for micromolded (107 kPa) and UV micropatterned hydrogel layers (52 kPa). For each MTF (muscle thin film), the twitch (difference between systolic and diastolic) stresses were calculated, averaged, and compared between pacing rates using a customized MatLab (MathWorks Inc., Natick, Mass.) script and One Way ANOVA run on SIGMAPLOT software (Systat Software, San Jose, Calif.).

### Example 2: Methods for Photopatterning Gelatin Hydrogels

Organ-on-chip technology combines approaches from cell biology, physiology, and tissue engineering with microsystems engineering and microfluidics to create a microphysiological environment of living cells that recapitulate human tissue and organ-level functions in vitro. The goal of organs-on-chips is to improve preclinical assays for drug safety and development by mimicking the physiology and pathophysiology of healthy and diseased human tissues. However, to become a next-generation tool for drug development and biomedical research in industry, organ-on-chips need to be amenable to large-scale continuous, automated, and quality-controlled fabrication, as opposed to the small-batch manufacture predominant in academic research. In particular, scalable fabrication strategies are needed for producing organ-specific 2D and 3D hydrogel extracellular matrix scaffolds that provide micromechanical cues for cellular adhesion, shape, differentiation, and cell-cell interactions. Cardiac and skeletal muscle organ-on-chip platforms exploit deformable hydrogel substrates with topographical micropatterns to achieve the physiological organization needed to test drug-induced toxicity [9], quantify tissue architecture, contractile function, and human cardiovascular diseases.

Many approaches for micropatterning hydrogels have been developed and include stereolithographic “bottom-up” methods that pattern structures through layer-by-layer fabrication or molding. Alternatively, “top-down” techniques involve the optical patterning of pre-formed hydrogels. One of the most versatile and common “bottom-up” methods is the direct molding of patterned hydrogel surfaces and requires a sequence of interdependent photolithography and casting steps. Current post-gelation optical patterning approaches can be done in a separate single step, but allow only for limited surface modifications. Common to most of these patterning approaches is their limited scalability or ease-of-use, meaning that they do not simultaneously allow for high-throughput automation while supporting a wide range of possible pattern dimensions.

As described herein, a new photopatterning method for ablating and micropatterning gelatin hydrogels using an ultraviolet (UV) laser has been developed. Specifically, a UVA-light activated photosensitizer (riboflavin-5′phosphate) and a UVA laser engraving system was adapted to photoablate the surface of uniform gelatin hydrogels and create anisotropic micropatterns suitable for tissue engineering and organ-on-chip applications. The novelty of the presented approach is that it enables maskless rapid micropatterning of a gelatin film without altering the hydrogel surface mechanics. The presented methods and results show that a novel tool for the automated and fast fabrication of micropatterned hydrogels for use in organ-on-chip applications has been developed. In contrast to the currently wide-spread method of mechanical molding of gelatin, this approach allows for scalable fabrication strategies enroute to mass manufacture and standardization of organ-on-chip platforms. Specifically, the new top-down photopatterning method shortened the time needed to manufacture gelatin substrates with a new pattern by 60% compared to traditional photolithography-based bottom-up approaches using direct micromolding. As a quality control for our fabrication method, the biocompatibility of UV-micropatterned gelatin for cardiac tissue engineering was validated by quantifying the viability, contractility, and sarcomeric structural orientation of neonatal rat and human iPS-derived cardiomyocytes (iPSCs). The ability to test novel patterns for single cell structural phenotyping of iPSCs was also evaluated. Finally, this fabrication method was tested as a rapid manufacturing process to produce engineered thin films used on our heart-on-a-chip platform and recapitulate appropriate contractile responses with neonatal rat cardiac tissues up to 27 days in culture.

### Soft Lithography Fabrication of Stamps for Micromolding Hydrogels

Elastomeric stamps were fabricated from polydimethylsiloxane (PDMS, Sylgard 184, Dow Corning, Midland, Mich.) using previously published protocols (Agarwal et al. Adv Funct Mater, 2013. 23(30): p. 3738-3746; Whitesides et al. Annual Review of Biomedical Engineering, 2001. 3(1): p. 335-373). In a cleanroom facility, silicon wafers (Wafer World, West Palm Beach, Fla.) were rinsed, air dried, and plasma treated to clean the wafer and introduce polar groups to the surface. Next, wafers were coated with SU-8 3005 photoresist (MicroChem, Newton, Mass.) on a spin-coater (Spincoat G3P-8, Specialty Coating Systems, Inc., Indianapolis, Ind.) and spun at 4000 rpm to generate wafers with 5 μm feature height. Using forceps, wafers were transferred to a level 65° C. hot plate for 30 seconds, then baked on a 95° C. hot plate for 2 minutes. After cooling for 1 minute, customized photomasks with 10 μm lines separated by 10 μm-wide transparent lines were placed on top of the wafers, secured, and exposed to 355 nm wavelength UV light using a mask aligner system (ABM, Scotts Valley, Calif.). Following UV exposure, wafers were baked on hot plates at 65° C. for 1 minute and 95° C. for 1 minute. The wafers were then rinsed and developed in propylene glycol monomethyl ether acetate (Thermofisher Scientific, Waltham, Mass.) for up to 5 minutes to dissolve un-exposed regions. Next, wafers with surface patterns were dried and coated overnight with silane (United Chemical Technologies, Bristol, Pa.) in a vacuum chamber. PDMS was poured onto the wafers, cured at 65° C. for at least six hours, carefully peeled from the wafer, and cut into stamps. These stamps featured 5 μm tall and 10 μm wide ridges spaced by 10 μm wide gaps that were used for micromolding gelatin hydrogels. The fabrication time of this method was compared with UV laser micropatterning methods described herein.

### Hydrogel Fabrication

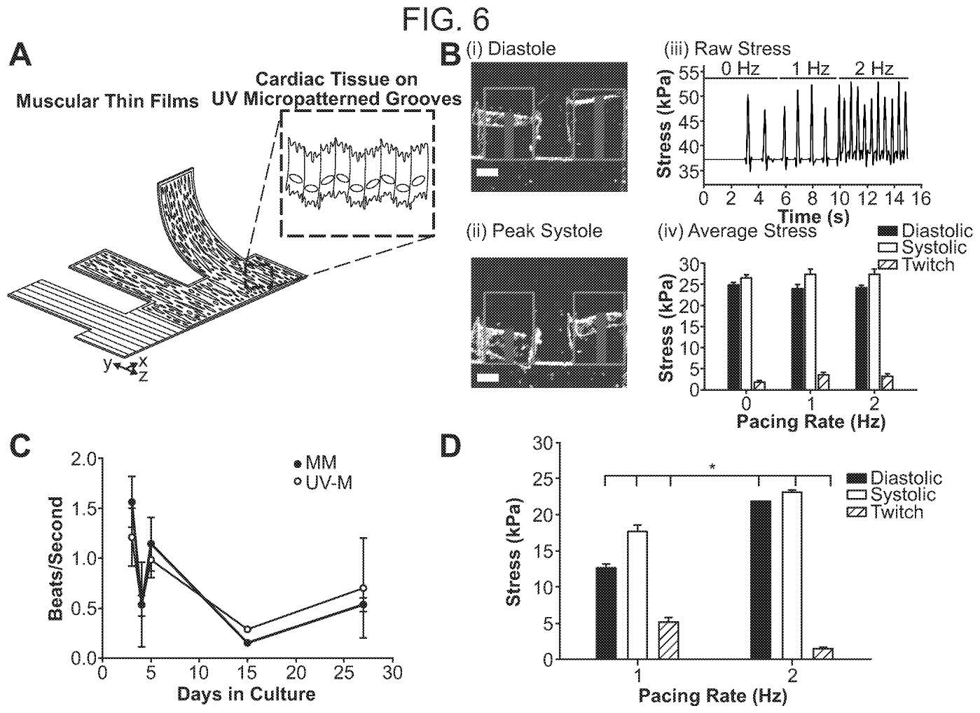

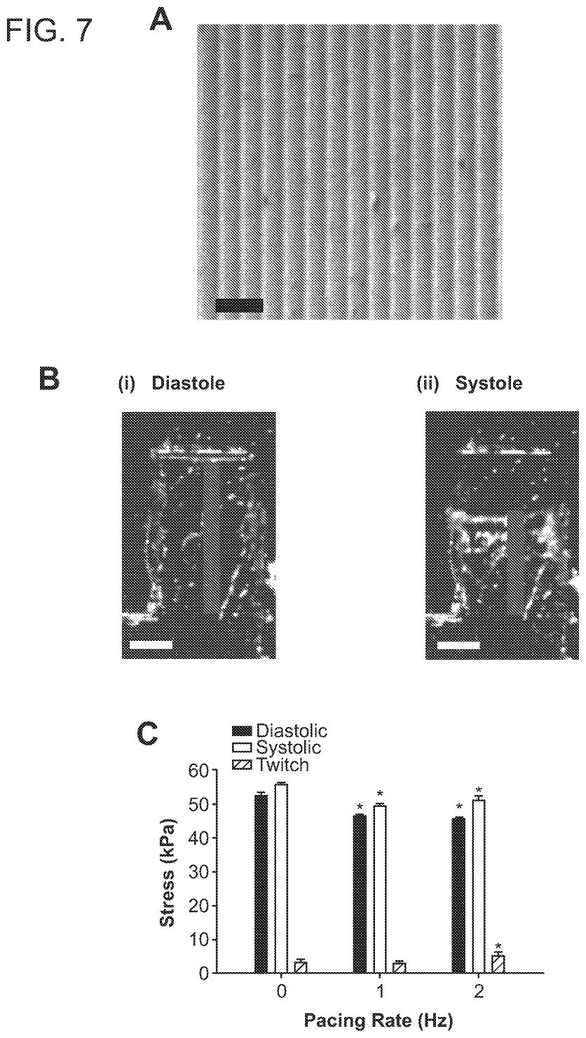

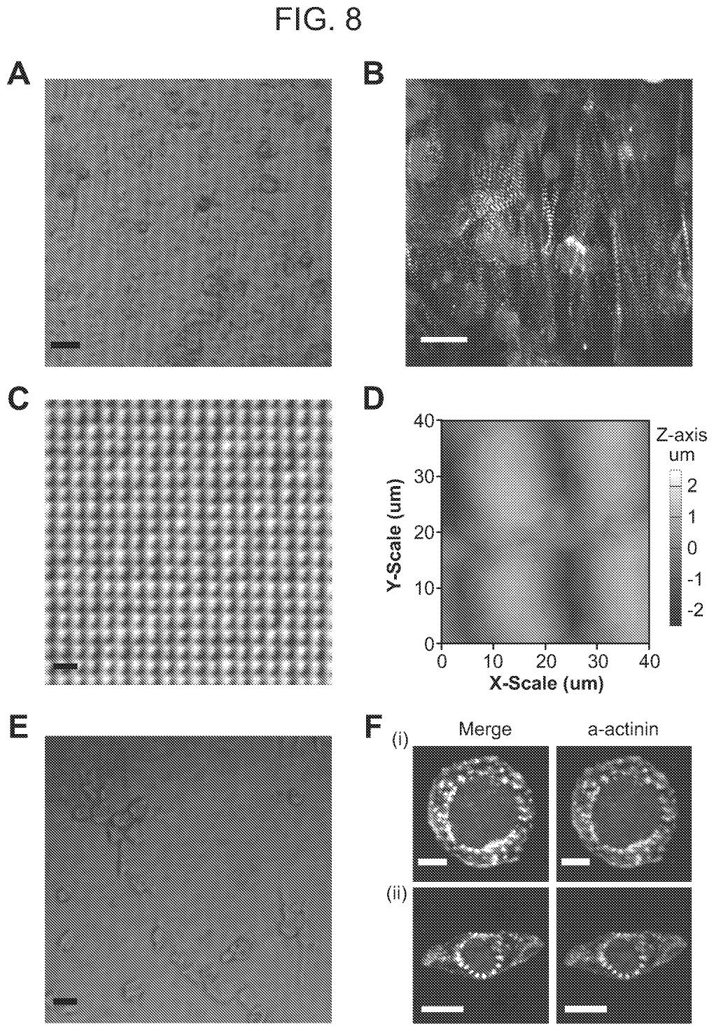

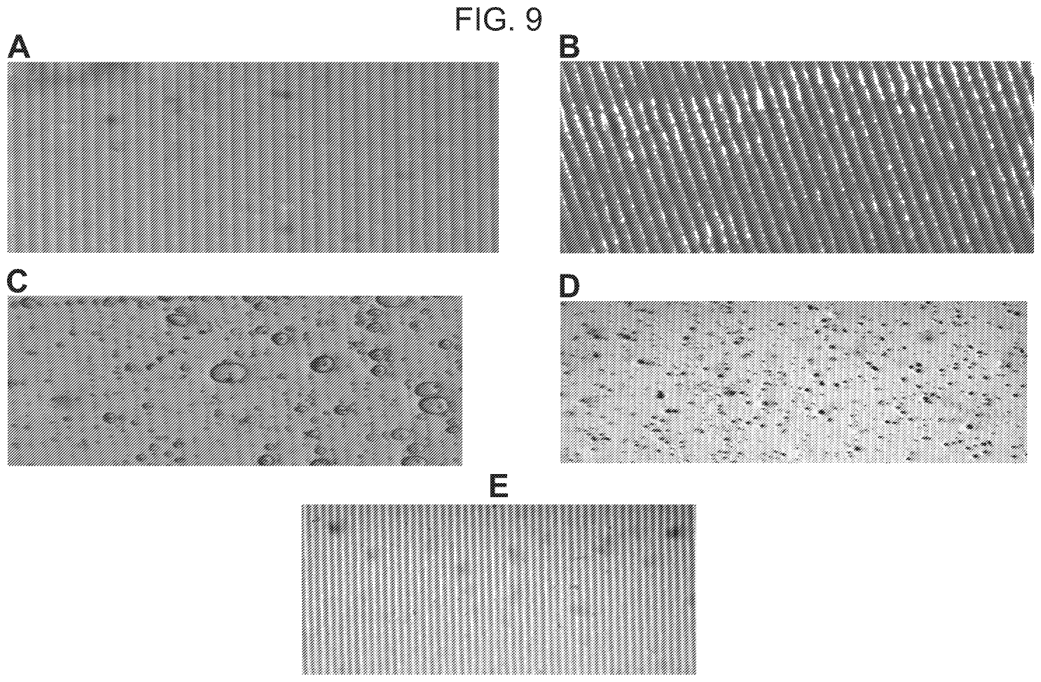

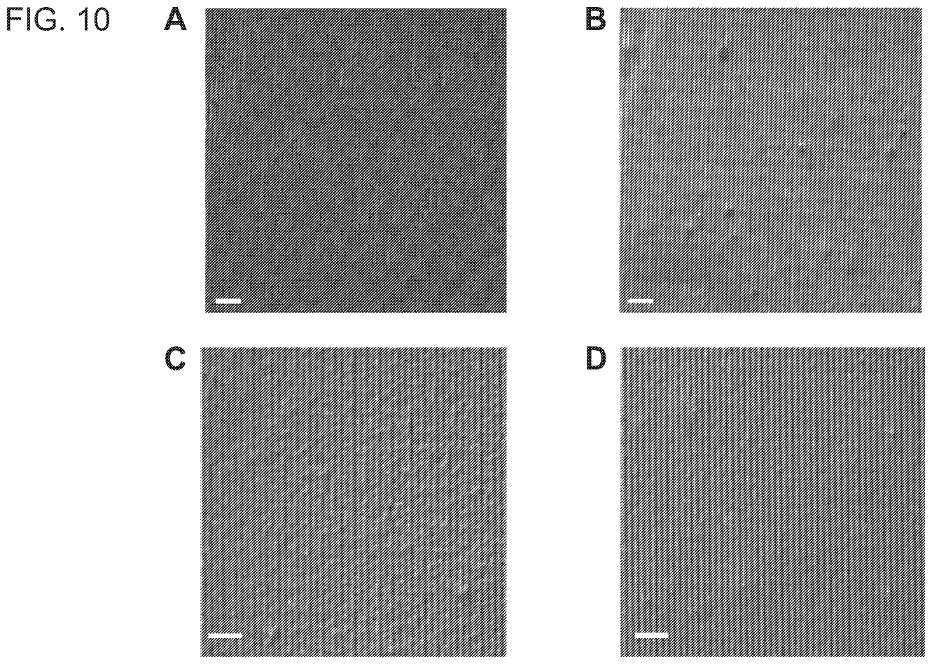

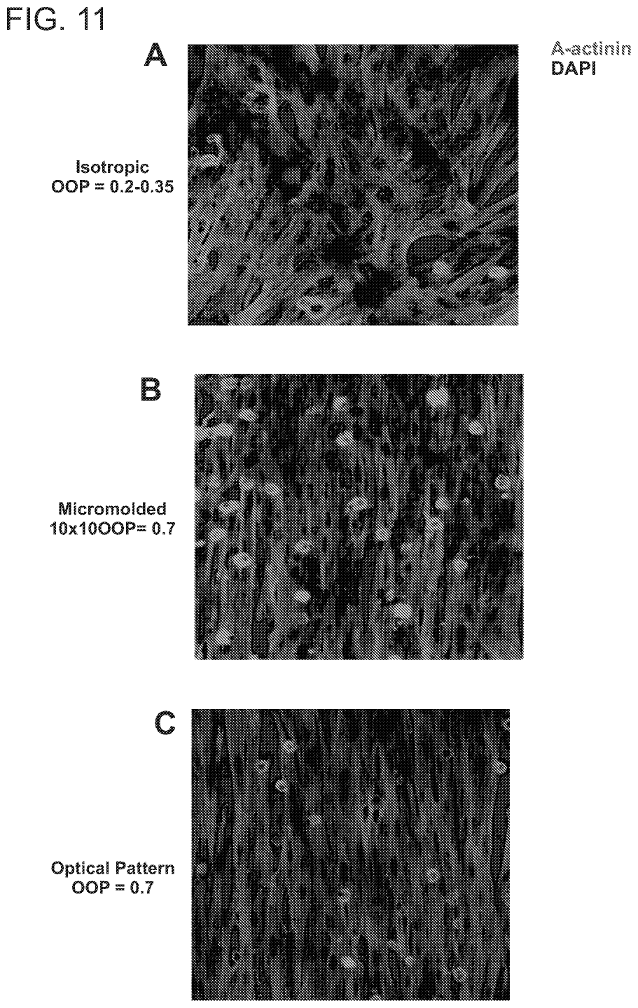

Cyclic olefin copolymer (COC) Topas® 5013-S04 laboratory slides (75 mm×25 mm×0.27 mm, Polylinks, Arden, N.C.) were masked with low adhesive tape (orange tape, 3M, St. Paul, Minn.) to provide boundaries for the hydrogels. The masking tape was cut with an LPKF UV laser engraving system (355 nm wavelength, LPKF Laser and Electronics, Tualatin, Oreg.) into 15×15 mm squares, for large tissues, or 18 mm diameter ellipses with an internal rectangular windows for muscular thin film fabrication. The tape was removed to expose squares and inner rectangles for the heart-on-a-chip in order to plasma treat the surface. COC slides were oxygen plasma treated for 5 minutes using a Plasma Prep III reactor (Structure Probe, Inc. West Chester, Pa.) to clean and introduce polar groups to the surface of the slides to allow for strong adhesion of gelatin (Beaulieu et al. Langmuir, 2009. 25(12): p. 7169-7176). To prepare the gelatin hydrogel, 20% w/v type A porcine gelatin (175 g bloom, Sigma-Aldrich, St. Louis, Mo.) was dissolved in distilled water at 65° C. Cross-linking agent, 8% microbial transglutaminase (mTG) (Ajinomoto, Fort Lee, N.J.) solution was warmed to 37° C., degassed in a vacuum chamber for 2 minutes, and heated back to 37.0 until fully dissolved. Equal parts of gelatin solution and mTG solution were mixed at a 1:1 ratio, resulting in a final concentration of 10% w/v and 4% w/v, respectively. A drop of gelatin solution was pipetted onto the COC slides and heart-on-a-chip substrates. Micromodled (MM) gelatin hydrogels were fabricated as previously described (supra), using a PDMS stamp with 10 μm by 10 μm line patterns. For UV-M (UV-micropatterned) and unpatterned (UN) gels, a dry glass microscope slide cleaned with 70% ethanol was gently lowered onto the gelatin droplet until stopped by the bounding of the masking tape. The tape acted as a spacer for controlling gel thickness (supra). The gelatin was cured overnight for 12 hours in a humidified Petri dish. Once cured, the gelatin was hydrated with water to prevent adhesion to the glass and the glass slide was carefully peeled off the gelatin. For UV micropatterning, hydrated gelatin surface was treated with 0.05% (w/v) riboflavin-5′phosphate (Sigma-Aldrich) for 10 minutes. Following treatment, the gelatin gels were rinsed with water and immersed in water for 10 minutes to remove all excess riboflavin-5′phosphate. The slides were dried with filtered air for at least 30 minutes in a customized drying chamber on low speed. Once samples were completely dry, hydrogels were patterned with the UV laser engraver. For experiments detailed in FIG. 6, muscle thin films (MTFs) were cut out using the UV laser engraver at higher power settings. All samples were rinsed overnight in phosphate buffer solution (Thermofisher Scientific). All solutions described were based in UltraPure DNAse/RNAse free distilled water (Thermofisher Scientific).

### UV Laser Micropatterning and Sample Preparation

Designs for cell patterning were created in CorelDraw graphic design software (Corel Inc., Ottawa, Canada) and exported into CircuitCam and CircuitMaster software (LPKF Laser and Electronics), respectively. Prior to laser cutting, a micrometer was used to measure gelatin and COC slide thickness for calibration of the laser beam focus. The Protolaser U3 laser engraver with a 15-μm beam diameter was used to engrave the gelatin with vector lines spaced by 22 μm (as measured from beam center point) and to cut MTF cantilevers (2 mm×1.3 mm) from the gelatin. For the generation of single cell islands, the same spacing in the vertical and horizontal alignment were employed to generate 7 μm by 7 μm square micropillars. Laser beam speed, also referred to as mark speed, was adjusted such that the distance between untreated surface and line trough (i.e., half the wave amplitude) was greater than 2 μm, as measured using confocal microscopy (Zeiss Axio Observer, Oberkochen, Germany). The following laser settings produced micropatterned lines for UV-M and micropillar (μ-pillar) hydrogels: power=0.13 to 0.16 Watts (W), frequency=50 kilohertz (kHz), mark speed=80 to 160 millimeters per second (mm/s), with 1 repetition. The following UV laser settings to cut through gels were used to produce MTF cantilevers: power=0.3 W, frequency=50 kHz, mark speed=50 mm/s, with 20 to 50 repetitions. Mark speed and repetitions for cutting cantilevers were altered according to gelatin thickness and verified using stereoscope microscopy (Leica Microsystems, Inc., Wetzlar, Germany) and Nikon 500 digital camera (Nikon, Tokyo, Japan). The remaining gelatin was removed from the MTF cantilevers using forceps. Cantilevers were manually lifted off the COC slide to fully detach from the plastic substrate prior to cell seeding. All MM and UV-M substrates were sterilized with 70% ethanol for 5 minutes, rinsed with sterile phosphate buffer solution, and left under the UV light of a standard sterile workbench for 5 minutes. Custom 8 mm thick acrylic rings (McMaster-Carr, Robbinsville, N.J.) were cut out with a laser engraving system (Epilog Laser, Golden, Colo.) to keep gelatin-coated COC slides from floating. These rings were also sterilized with 70% ethanol for 5 minutes, dried with air, and UV ozone-treated for 5 minutes in a UV ozone cleaner (Jelight Company, Inc, Irvine, Calif.). The rings were placed onto the gelatin-COC slides in a 6-well cell culture plate to direct cells onto the gelatin surface during seeding. All substrates were hydrated in sterile phosphate buffer solution until cell seeding. This method reduces the time required to generate micropatterned gelatin by 60% compared to traditional micromolding and soft lithography methods.

Fibronectin Crosslinking of Gelatin for Human iPSC Cellular Attachment

For human iPS-derived cardiomyocyte (iPSC) experiments, pre-patterned gelatin hydrogels were crosslinked with fibronectin to aid with cellular attachment. First, 3.42 g sodium acetate (Sigma Aldrich) was dissolved in 400 mL deionized water and titrated with acetic acid and NaOH (Sigma Aldrich) monitoring pH until it reached 5.5. Sodium acetate buffer was then filtered in a sterile cell culture hood. The 0.4 mg/mL 1-ethyl-3-(3-dimethylaminopropyl)carbodiimide hydrochloride (EDC, ThermoFisher Scientific) and 1.1 mg/mL sulfo-N-hydroxysuccinimide (NHS, Sigma) were dissolved in fresh sodium acetate buffer in separate conical tubes. In the cell culture hood, EDC and NHS solutions were filtered with a 0.2 um syringe filter for sterility. Next, 10 μl of EDC and 10 μl of NHS solutions were added to a 100 μl aliquot of sterile 1 mg/mL fibronectin (BD Biosciences, San Jose, Calif.) and incubated for 15 minutes. After the 15 minute incubation period, the entire EDC-NHS-fibronectin solution was diluted in sterile phosphate buffer solution for a final fibronectin concentration of 50 μg/mL and added to the surface of the gelatin hydrogels. Hydrogels were incubated in EDC-NHS-fibronectin solution for 2 hours at room temperature. Following incubation, gels were rinsed with fresh phosphate buffer solution three times and prepared for cell seeding.

### Atomic Force Microscopy Imaging

Atomic force microscopy (AFM) imaging was performed using MFP-3D AFM system (Asylum Research, Santa Barbara, Calif.) with an open fluid droplet containing deionized water. The COC-gelatin slides were fixed to glass slides using carbon tape and sample bond adhesive (Ted Pella, Redding, Calif.) for mounting on the AFM stage. Prior to hydrogel contact, AFM cantilevers were calibrated in air and water using the Sader method to ensure reliable topography and elastic modulus measurements (Review of Scientific Instruments, 1999. 70(10): p. 3967-3969). All topography images for hydrated hydrogels were collected in contact mode with soft, gold-coated silicon nitride bio-levers (Olympus TR400PB, Asylum Research Probe Store, Santa Barbara, Calif.) with a constant contact force ranging from 1 to 10 nN to prevent adhesion. After collecting a contact mode image of each gel sample in water, the tip was placed on three different sites on either the micropatterned ridges or troughs and at least 25 force distance curves (FDCs) were collected from each site. The scan rate (0.8 Hz) and distance traveled (1.5 μm) were kept constant for each FDC. All FDCs were analyzed using the Johnson-Kendall-Roberts (JKR) model (Proceedings of the Royal Society of London. A. Mathematical and Physical Sciences, 1971. 324(1558): p. 301-313) built into the instrument software to estimate the elastic modulus of the hydrogels.

### Cell Culture

Neonatal rat ventricular myocytes (NRVMs) were isolated from 2-day old neonatal Sprague-Dawley rats according to protocols approved by the Harvard University Animal Care and Use Committee. After isolation, ventricles were homogenized in Hanks balanced salt solution followed by overnight trypsinization and digestion with collagenase at 4.0 (1 mg/mL, Worthington Biochemical Corp., Lakewood, N.J.). Cell solutions were filtered and re-suspended in M199 culture medium supplemented with 10% heat-inactivated fetal bovine serum (ThermoFisher Scientific), 10 mM HEPES, 0.1 mM MEM nonessential amino acids, 20 mM glucose, 2 mM L-glutamine, 1.5 mM vitamin B-12, and 50 U/mL penicillin (Sigma-Aldrich). Cells were pre-plated twice to reduce non-myocyte cell populations. Neonatal rat cardiac myocytes were seeded onto hydrogel substrates at a density of 2000 cells/mm2. Cell culture medium was exchanged to maintenance medium containing 2% fetal bovine serum (FBS) every 48 hours (McCain Biomaterials, 2014, 21:5462-71).

For experiments with human iPS-derived cardiomyocytes (iPSCs, Axiogenesis, Cologne, Germany), cells were thawed from vials and plated in a 6-well culture dish in Cor.4U medium according to manufacturer's protocols. Two days prior to cell seeding onto UV-M line gels and micropillars (μ-pillars), cells were trypsinized with 0.25% trypsin-EDTA (ThermoFisher Scientific) for 5 minutes at 37° C. and washed three times with warm Cor.4U medium. All cell culture medium was collected into a 15 mL conical tube and centrifuged at 200×g for 5 minutes. The supernatant of the medium was aspirated to leave a pellet of human iPSCs. Cells were then re-suspended with 0.5 mL of Cor.4U medium and 20 μl of solution was removed for cell counting (supra). The tube of cells were kept at 37° C. while cell counting was performed using a standard hemocytometer. After cell counting, human iPSCs were dispersed onto line micropatterns at a seeding density of 2000 cells/mm² for tissues and a seeding density of 600 cells/mm² for single cell islands. Human iPS-derived cardiomyocytes were cultured for 9 days with media changes every 48 hours prior to fixation.

### Immunostaining and Structural Analysis

Engineered cardiac tissues were pre-fixed with warm 2% paraformaldehyde (Electron Microscopy Sciences, Hatfield, Pa.) for 2 minutes, then fixed with fresh 4% paraformaldehyde and 0.05% Triton-X (Sigma-Aldrich) for 8 minutes. Tissues were gently washed three times with phosphate buffer solution and incubated with 5% (w/v) bovine serum albumin (BSA, Sigma-Aldrich) for 30 minutes. Next, tissues were then incubated with primary antibodies against sarcomeric α-actinin (1:200, Sigma-Aldrich), DAPI (1:200, Invitrogen, Carlsbad, Calif.) and Alexa Fluor 546 phalloidin (1:200, Invitrogen) for 60 minutes at room temperature. After the 60 minute incubation, tissues were gently rinsed three times for 5 minutes each with 0.5% BSA in phosphate buffer solution. Tissues were incubated with secondary antibodies against mouse IgG conjugated to Alexa Fluor 488 (1:200, Invitrogen) for 60 minutes. All antibodies described were diluted in 0.5% BSA and 200 μl of solution was added to each tissue. Plates were covered with aluminum foil during incubation steps. Following incubation with secondary antibodies, tissues were gently rinsed three times with 0.5% BSA prior to mounting on glass slides. Stained hydrogels were mounted tissue side up on glass microscope slides and treated with Prolong Gold Anti-Fade reagent (Thermofisher Scientific). A 22 mm×22 mm square glass slide was placed over the top of the hydrogel and left to dry overnight in a dark protected chamber prior to sealing the slide with nail polish. Slides were imaged using a Zeiss Axio Observer inverted confocal microscope (Zeiss) with 40× and 60× glycerol objectives. For each slide, multiple images with different fields of view were analyzed to determine the anisotropy and sarcomeric orientation of the engineered cardiac tissues and single cells. Custom algorithms implemented in ImageJ and Matlab (The MathWorks Inc., Natick, Mass.) were used to compute the orientation angles of the lattice structure of sarcomeric α-actinin and the resultant vectors were used to calculate the total orientational order parameter (OOP). The OOP is defined as the mean resultant vector from the frequency distribution of α-actinin orientations that has been previously described by the laboratory and has been commonly used to describe liquid crystals (Pasqualini et al. Stem Cell Reports, 2015. 4(3): p. 340-347; Sheehy et al. Stem Cell Reports, 2014. 2(3): p. 282-294; Volfson et al. Proceedings of the National Academy of Sciences, USA 2008. 105(40): p. 15346-15351; Kuczyński et al. Molecular Crystals and Liquid Crystals, 2002. 381(1): p. 1-19). Here, the OOP is used for quantifying cardiac tissue alignment, where a value of 0 indicates an isotropic orientation and a value of 1 represent perfectly aligned sarcomeres (Grosberg et al. Lab Chip, 2011. 11(24): p. 4165-730). The OOPs for each condition were averaged and statistically compared using Kruskal-Wallis one way ANOVA and Dunn's test or Student's t-test. To determine the orientation and packing density of sarcomeric α-actinin in single human iPSCs, sarcomeric packing density (SPD) was computed using custom ImageJ and Matlab (The MathWorks Inc.) algorithms described previously (Schneider et al. Nat Methods, 2012. 9(7): p. 671-675). The SPD used here is computed as the fraction of immunosignal that is localized in a regular lattice at the distance of the sarcomere. Using a scoring system for maturation of the iPSC cytoskeleton, a score of 0 represents diffuse sarcomeric α-actinin staining and poor orientation, while a score of 1 represents a highly organized lattice of sarcomeric α-actinin. Then SPD values were compared with that of previously published results on microcontact-printed substrates (Czerner et al. Procedia Materials Science, 2015. 8: p. 287-296).

### Muscular Thin Film Experiments and Analysis

Muscular thin film (MTF) experiments were performed as previously described (McCain et al. Biomaterials, 2014, 21:5462-71). After 5 days in culture, heart chips were rinsed and immersed in a 60 mm Petri dish filled with warm Tyrode's solution (37° C.) prior to video recording MTFs. The Tyrode's solution contained (mM): 135 NaCl, 1.8 CaCl2, 5 KCl, 1.0 MgCl2, 5 HEPES, 5 D-glucose, and 0.33 NaH2PO4 (Sigma-Aldrich). Next, cardiac tissues were imaged under a stereomicroscope (Model MZ6 with darkfield base, Leica Microsystems, Inc.) and all thin film cantilevers were peeled off the plastic carrier with forceps. To remove debris, tissues were transferred to a new 60 mm Petri dish with fresh Tyrode's solution at 37° C. A custom lid with platinum pacing electrodes was attached to the top of the Petri dish and connected to an electrical pulse generator (MyoPacer Cell Stimulator, IonOptix, Milton, Mass.) to deliver field stimulation at 1-2 Hz and 5-10 V with a square wave pulse of 10 millisecond duration (Zhang et al. Proceedings of the National Academy of Sciences, USA 2017. 114(12): p. E2293-E2302; McCain et al. Biomaterials, 2014, 21:5462-71). Video recordings of both spontaneously contracting and paced MTF tissues were performed using a Basler area scan camera (100 frames per second, 1920×2000 pixels, Basler, Exton, Pa.) mounted to the stereomicroscope. Movies were imported into a customized tracking software, MTF Video Processor (MVP), to measure cantilever displacement. The MVP software performs frame-by-frame processing to subtract the background, isolate the MTFs, detect the MTF edges, and use frame-by-frame subtraction to detect the edge displacement in x-projection as a function of time. The x-projections and corresponding time points of each movie were imported into a Microsoft Excel (.csv) file for further analysis. Given the x-projection data and original length of the cantilevers, the radius of curvature as a function of time was calculated for each MTF in Matlab (Alford, P. W. et al. Biomaterials, 2010. 31(13): p. 3613-21). The radius of curvature, thickness, and elastic modulus of each MTF were then used to calculate stress using a modified Stoney's equation (Grosberg et al. J Pharmacol Toxicol Methods, 2012. 65(3): p. 126-35).

The modified Stoney's equation is as follows:

$$σ = \frac{{Et}_{s}^{2}}{6 ( 1 - {v}^{2} ) {Rt}_{c} ( 1 + \frac{{t}_{c}}{{t}_{s}} )}$$

where σ is the contractile stress exerted by the cardiac muscle layer, E is elastic modulus of the gelatin, ts is gelatin thickness, R is MTF radius of curvature, tc is the thickness of the cardiac muscle tissue, and D is Poisson's ratio for an incompressible solid (0.5) (Czerner et al. Procedia Materials Science, 2015. 8: p. 287-296). Here, the elastic modulus was derived from the bulk stiffness modulus previously reported for gelatin hydrogels (55 kPa) (McCain et al. Biomaterials, 2014, 21:5462-71). As measured by confocal microscopy, gelatin film thickness was 180 μm, which is in agreement with previous studies. Gelatin film thickness is determined by the thickness of the orange tape used in the fabrication process, as the tape serves as boundary and spacer for the gelatin cast onto the COC slides. Myocardium thickness was ˜8 μm as measured using confocal microscopy. For each MTF, the average twitch stresses (difference between systolic and diastolic stress) for different pacing rates were calculated (Table I). Statistical significance was determined by Kruskal-Wallis one way ANOVA and Dunn's test using SigmaPlot software (Systat Software, San Jose, Calif.).

TABLE I
Muscular thin film contractile stress.

| | Pacing | | | | |
|---|---|---|---|---|---|
| | Rate | Diastolic | Systolic | Twitch | |
| | (Hz) | Stress (kPa) | Stress (kPa) | Stress (kPa) | N= |
| UV Laser | 0 | 24.7 ± 0.6 | 26.5 ± 0.7 | 1.8 ± 0.3 | 12 films, |
| Micropatterned | | | | | 6 chips |
| Gelatin 5 Days | 1 | 23.4 ± 0.8 | 26.5 ± 1.0 | 3.1 ± 0.4 | 13 films, |
| | | | | | 6 chips |
| | 2 | 24.1 ± 0.7 | 27.3 ± 1.2 | 3.3 ± 0.5 | 9 films, |
| | | | | | 5 chips |
| UV Laser | 1 | 12.6 ± 0.6 * | 17.7 ± 0.8 * | 5.1 ± 0.6 | 3 films, |
| Micropatterned | | | | | 1 chip |
| Gelatin 27 Days | 2 | 21.8 ± 0.01 ^{#} | 23.2 ± 0.1 ^{#} | 1.5 ± 0.2 ^{#} | 2 films, |
| | | | | | 1 chip |

* P < 0.05 vs UV laser micropatterned gels at 5 days, same pacing rate
^{#} P < 0.05 vs. 1 Hz pacing at 27 days

### Results and Discussion

### UV Laser Micropatterning of Gelatin Hydrogels

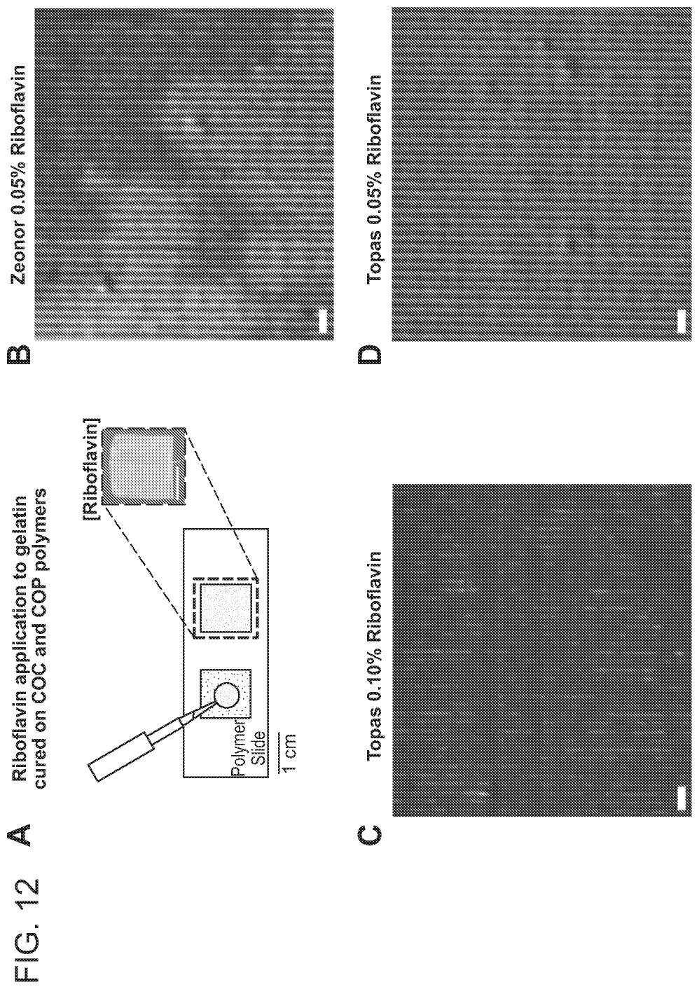

Here, engineered micropatterned hydrogels were generated for tissue engineering and organ-on-chip applications without the use of soft lithography or mechanical molding. For this, a protocol for casting and adhering a thin gelatin film to a polymeric laboratory slide (see, e.g., FIG. 1 and FIG. 2) was generated. The objective was to identify a robust, biocompatible plastic carrier that would replace commonly used fragile glass slides (McCain. Biomaterials, 2014, 21:5462-71), allow for controllable gelatin adhesion, and support optical imaging. The cyclic olefin polymer (COP) Zeonor® 1420R, cyclic olefin copolymer (COC) Topas® 5013-S04 (both from Polylinks, Arden, N.C.), as well as the polyolefin Permanox (Sigma-Aldrich) were tested for these properties. Oxygen plasma treatment of these materials was found to enable robust adhesion of gelatin thin films to either carrier material (FIG. 12) (Diaz-Quijada et al. Lab on a Chip, 2007. 7(7): p. 856-862; Sultanova et al. Optical and Quantum Electronics, 2013. 45(3): p. 221-232; van Midwoud, et al. Analytical Chemistry, 2012. 84(9): p. 3938-3944), whereas gelatin was easily removable from non-treated carriers, or other polymer substrates, such as acrylic and polycarbonate. A method to micropattern the gelatin films with a UV laser engraving system was subsequently developed (FIG. 1 and FIG. 2). Importantly, the 15 μm-wide UV beam diameter enabled the design and generation of patterns at scales similar to lithography-based micromolding (i.e., at the order of 1-20 μm) to mimic the anisotropic collagen-rich networks that guide cardiac tissue alignment in the ventricular myocardium (Gazoti Debessa et al. Mechanisms of Ageing and Development, 2001. 122(10): p. 1049-1058; Capulli et al. Advanced Drug Delivery Reviews, 2016. 96: p. 83-102).

UV-M parameters and consistency were found to depend on the concentration of photosensitive agent, type of plastic carrier, and laser engraver speed. Regarding the first factor, it was necessary to pre-treat the gelatin substrates with appropriate concentrations of photosensitive riboflavin 5′-phosphate (FIGS. 1B and 1D). Optimal riboflavin pre-treatment concentration (0.05% w/v) allowed for UV laser micropatterning of gelatin and generated uniform line patterns once laser parameters are calibrated (FIG. 12D). Higher riboflavin concentrations required re-adjustment of the laser parameters to avoid burning (FIGS. 12A and 2C), whereas omission of riboflavin treatment led to formation of burn marks, bubbles and irregular surface patterns at all laser settings. For untreated gelatin hydrogels, the UV laser was only suitable for through-cuts. Second, it was necessary to use a specific carrier composition. UV micropatterning of gelatin cast onto Zeonor® COP or Permanox polyolefin resulted in partial micropatterning and occasional burning of the gelatin surface (FIG. 12B). This is likely due to inherent differences in surface chemistry or optical properties between Zeonor®, Permanox®, and Topas® (Diaz-Quijada. Lab on a Chip, 2007). Third, the UV laser parameters for speed, power, and frequency were calibrated to achieve feature spacing, height, and width comparable to MM substrates for cardiac tissue engineering, as detailed in the methods. Thus, taking these factors into account, a reliable protocol for UV micropatterning of gelatin hydrogels was developed for use in tissue engineering and organ-on-chips applications.

### Micromechanics of Micromolded and UV Laser Micropatterned Gelatin Hydrogels

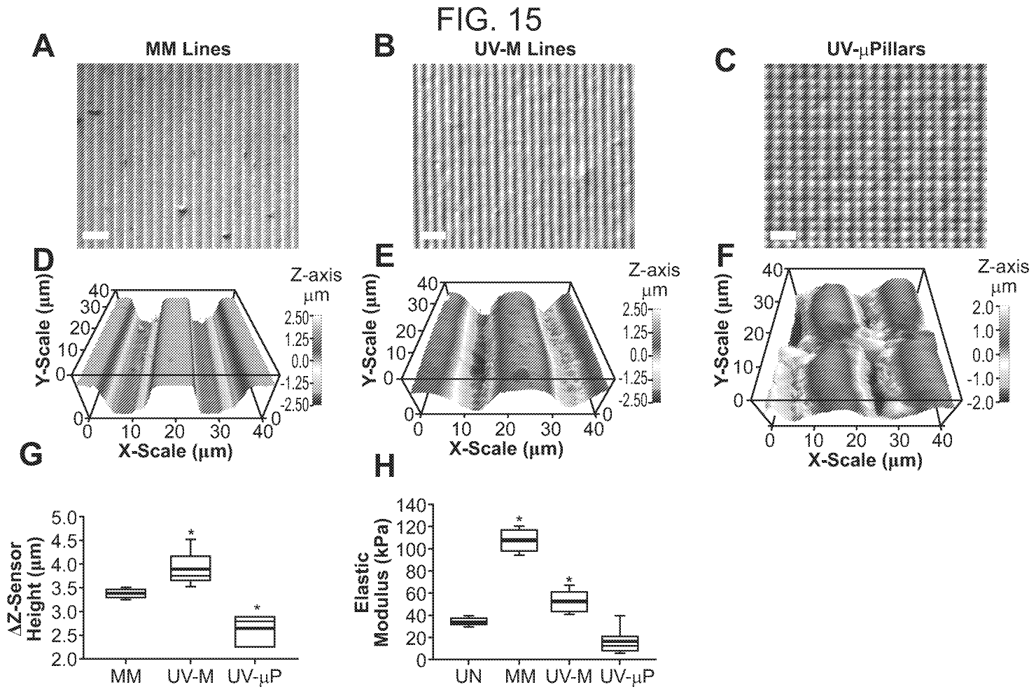

Heart-on-a-chip platforms aim to recapitulate the microenvironment of the human heart, including the elastic modulus (15 kPa) and laminar tissue structure (Wang et al. Nat Med, 2014; McCain. Biomaterials, 2014, 21:5462-71). To test the flexibility and rapid prototyping capabilities of our UV laser micropatterning approach, engineer gelatin lines for cardiac tissue alignment and single-cell gelatin micropillars (μ-pillars, UV-μP) were developed for human iPSC structural phenotyping. Additionally, the present method was compared to traditional molding techniques by fabricating 10 μm by 10 μm PDMS stamps for micromolded (MM) gelatin to generate micropatterned gelatin lines (FIG. 15A). Then, the UV-micropatterning fabrication approach described in FIG. 1 was used to design and fabricate 15 μm by 7 μm spaced lines for UV-M gelatin (FIG. 15B), and 7 μm by 7 μm spaced squares to create UV-μP gelatin islands (FIG. 15C). To further investigate the biomechanics of hydrated UV-micropatterned gelatin compared to unpatterned (UN) and MM hydrogels, atomic force microscopy (AFM) was used to determine the topographical and elastic properties of the hydrogel surface for each condition (Agarwal et al. Lab Chip, 2013. 13(18): p. 3599-608).

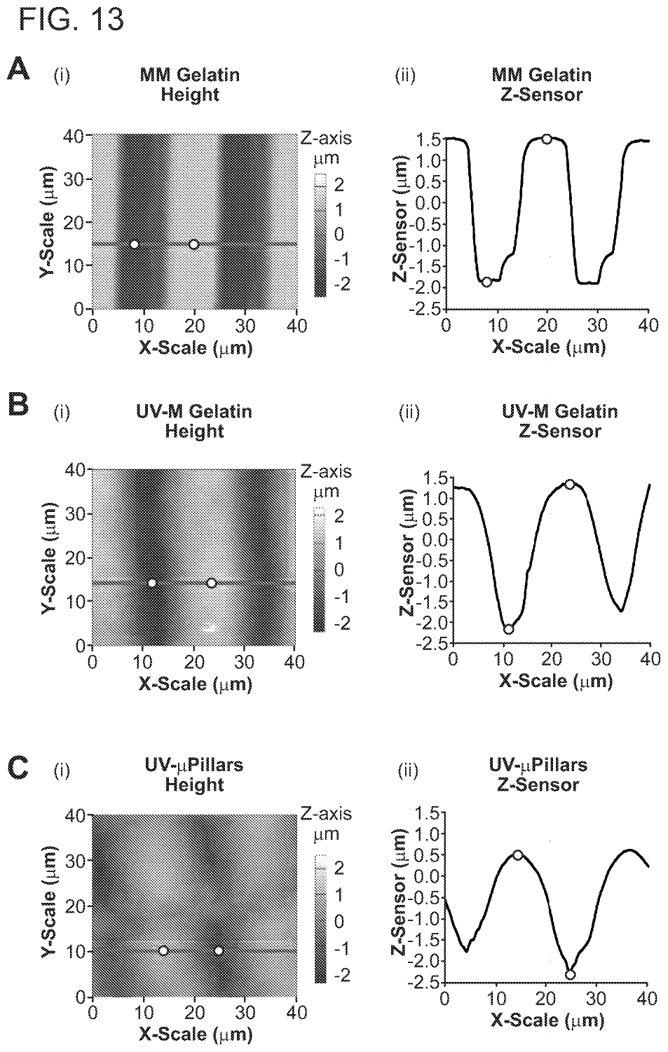

The grooves of MM gelatin hydrogels cast with PDMS stamps exhibit a square wave cross-section (FIG. 15D and FIG. 13A). By contrast, the grooves of UV-M gels exhibit a smoother, sigmoidal cross-section (FIG. 15E and FIG. 13B). Both UV-M hydrogels (mean height 3.9±0.1 μm, n=13, 4 samples) and MM hydrogels (3.4±0.02 μm, n=13, 4 samples) exhibit comparable feature heights within less than a micron from each other in peak to trough features (FIG. 15G and FIGS. 13A and 13B). The standard deviation of UV-M gelatin Z-sensor height is 0.3 μm, indicating that fabrication of these features are reproducible enough for large scale manufacturing of hydrogels for tissue engineering.

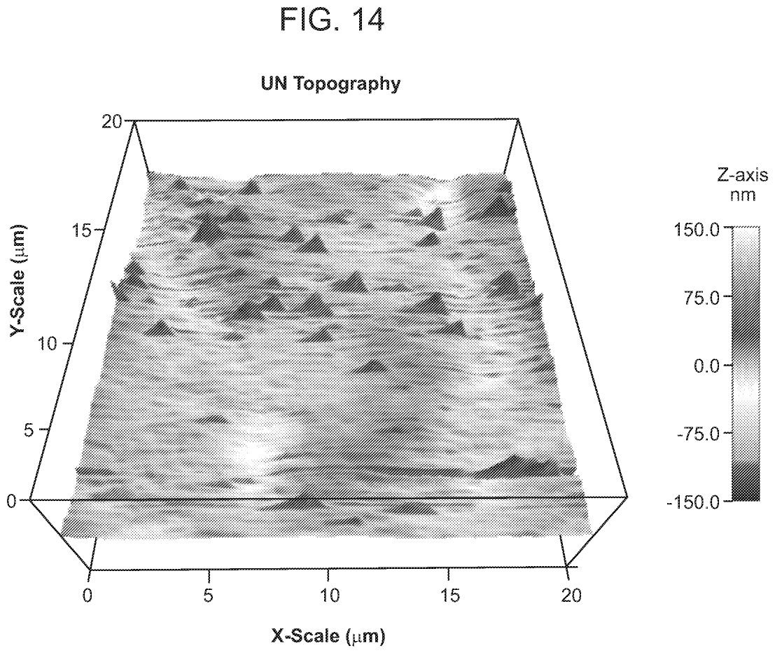

Atomic force microscopy also revealed that when UV-μP gels are hydrated, they expand to a 20 μm width from trough to trough (FIG. 15F) and exhibit a mean height of 2.6±0.3 μm, (n=3, 1 sample, FIG. 15G and FIG. 13C). As a control, UN hydrogels was casted onto COC slides and performed AFM topography measurements. From these measurements it was found that the topography of UN gelatin does not vary by more than 150 nm over a 20 μm2 area, ruling out substantial effects on the later UV-M topography (FIG. 14).

In addition to measuring surface topography, the elastic modulus of UN, MM, UV-M, and UV-μP gelatin were compared using AFM force distance measurements in liquid to identify the impact of these micropatterning methods on substrate rigidity (FIG. 15H). All these gels contained 10% w/v gelatin and were crosslinked with 4% microbial transglutaminase. At least 25 force distance measurements were performed at three independent sites on the top (crests) and bottom (troughs) of the hydrogels and calculated the average elastic modulus using a Johnson-Kendall-Roberts model. UN gelatin was found to exhibit an elastic modulus of 33.2±0.4 kPa (n=88 force distance curves). MM hydrogels exhibited an elastic modulus of 107.3±0.9 kPa (n=131 force distance curves) which is consistent with previous results (Bettadapur et al. Scientific Reports, 2016. 6: p. 28855). Interestingly, the MM elastic modulus is significantly higher than the elastic modulus of UN gels. Without being bound to any one particular theory, this finding suggests that the mechanical casting of the patterns causes an increase in surface stiffness during curing. Furthermore, UV-M hydrogels exhibit an elastic modulus of 52.4±0.7 kPa (n=180 force distance curves), which is significantly higher compared to UN gels, yet significantly lower compared to MM gelatin (P<0.05). Moreover, with respect to surface stiffness, UV-M substrates are more similar to UN gelatin than MM substrates. Finally, the elastic modulus at the top of the UV patterned μ-pillars was measured, where it was anticipated for cells to attach in subsequent experiments, to determine if patterning altered the surface modulus. AFM force distance measurements of UV-μP yielded an average modulus of 16.3±1.1 kPa (n=188 FDCs), which is lower than the elastic modulus of UN hydrogels and UV-M lines (not significant).

In summary, the surface elastic modulus of UV micropatterned hydrogels is on the same order of magnitude as the elastic moduli of human and rat heart in vivo (15 kPa) (Berry et al. American Journal of Physiology-Heart and Circulatory Physiology, 2006. 290(6): p. H2196-H2203; Bhana et al. Biotechnology and Bioengineering, 2010. 105(6): p. 1148-1160). Moreover, UV-M and UV-μP hydrogels exhibit a smooth, sigmoidal surface topography with suitable dimensions for cardiac tissue engineering and single cell islands. Using this photopatterning approach, microscale surface groove and pillar structures were generated with maximum feature height variation of 0.3 μm, demonstrating robustness and reproducibility.

Cardiac Tissue Engineering of Neonatal Rat Ventricular Myocytes with UV Laser Micropatterning

Following the fabrication and mechanical characterization of UV micropatterned hydrogels, UV-M, like traditional MM substrates, was anticipated to guide engineered tissue structure into recapitulating the anisotropic architecture of ventricular musculature on a 2-dimensional level. Therefore, UV-M, MM, and UN gelatin substrates were seeded with neonatal rat ventricular cardiomyocytes (NRVMs), and the expression and orientation of contractile proteins involved in myofibrillogenesis and contractile function were investigated (Dabiri et al. Proceedings of the National Academy of Sciences, USA 1997. 94(17): p. 9493-9498).

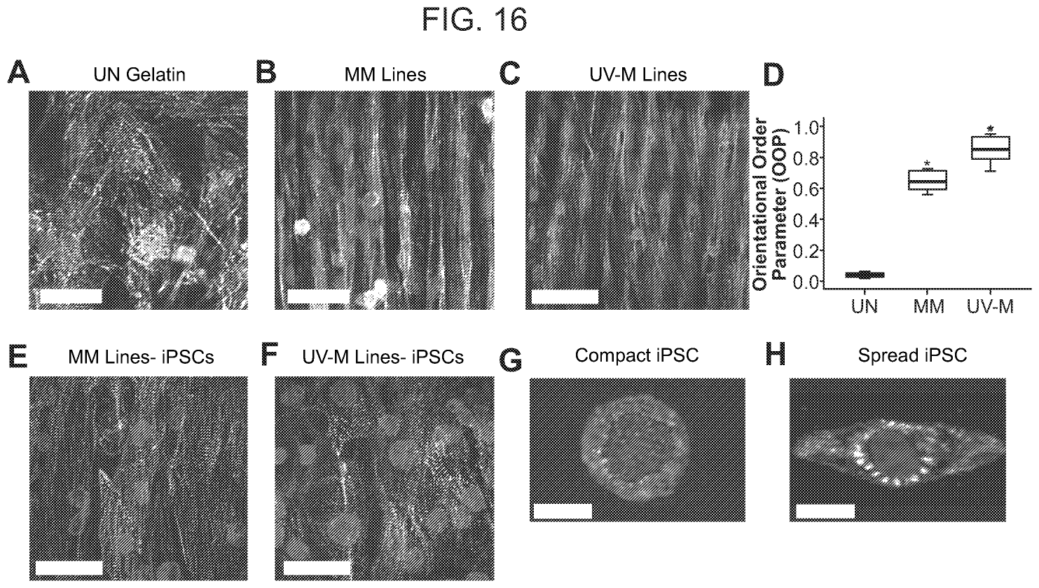

Here it was shown that NRVMs seeded on UV-M substrates formed anisotropic monolayers similar to those observed for MM hydrogels (FIGS. 16B and 16C). This is in stark contrast to NRVMs seeded on UN hydrogels (FIG. 16A). After 5 days in culture, the NRVM tissues formed on collagen-based hydrogels were fixed and immunostained for sarcomeric α-actinin to investigate the expression and structural organization of contractile proteins (FIG. 16A-16C). Sarcomeric α-actinin is essential for stabilizing the contractile apparatus of muscle tissues by localizing to the Z-disk of cardiomyocytes where it forms a lattice-like structure perpendicular to actin filaments (Bray et al. Biomaterials, 2010. 31(19): p. 5143-50). Previous studies have shown that the orientation of sarcomeric α-actinin is representative of cardiomyocyte maturity and cardiac tissue alignment on the tissue constructs (Grosberg et al. Lab Chip, 2011. 11(24): p. 4165-73; Pasqualini et al. Stem Cell Reports, 2015. 4(3): p. 340-347; Rodriguez et al. Journal of Biomechanical Engineering, 2014. 136(5): p. 0510051-05100510).

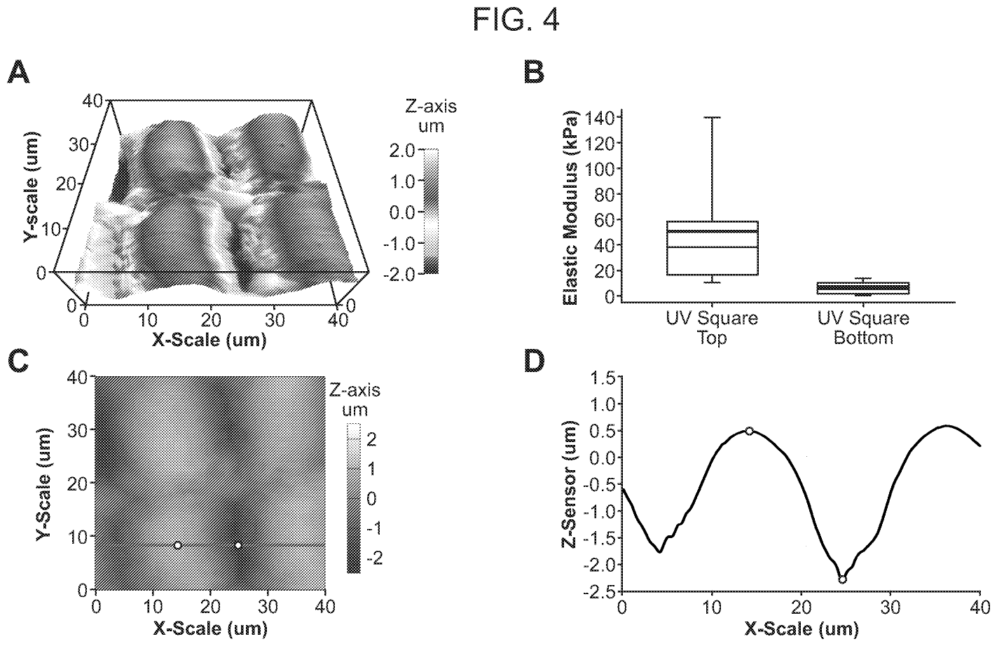

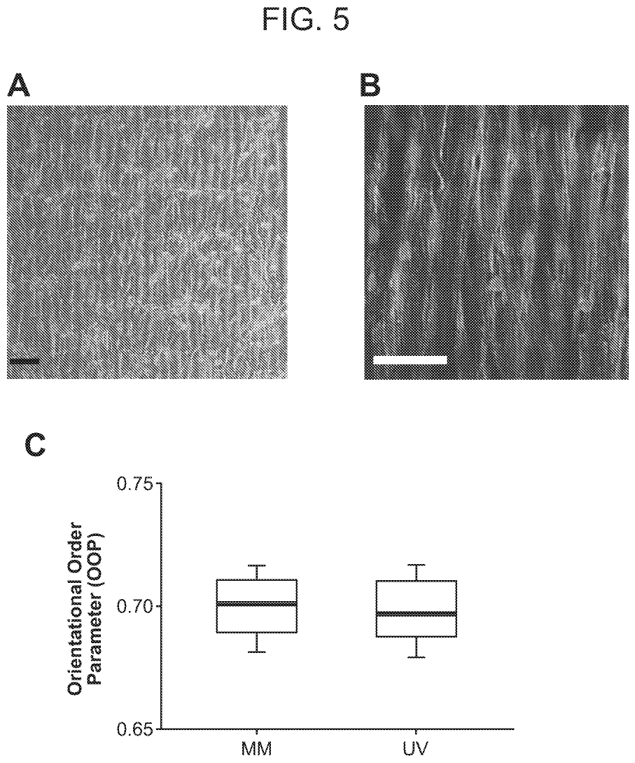

To quantify the degree of anisotropy, the total orientational order parameter (OOP) of sarcomeric α-actinin from immunostained images was computed. This parameter ranges from 0 (random organization) to 1 (perfect alignment) as a scoring system for cardiomyocyte tissue anisotropy (Pasqualini et al. Stem Cell Reports, 2015. 4(3): p. 340-347; Sheehy et al. Stem Cell Reports, 2014. 2(3): p. 282-294). As expected, NRVM tissues engineered on plain gelatin surfaces (UN) formed isotropic monolayers of cells with an OOP of 0.04±0.004 (n=8 images, 3 slides). Cardiac tissues engineered on MM gelatin achieved a significantly higher OOP of 0.65±0.01 compared to UN gels (n=24 images, 3 slides), which is consistent with previous studies (FIG. 4D) (McCain. Biomaterials, 2014. 21:5462-71; Agarwal et al. Adv Funct Mater, 2013. 23(30): p. 3738-3746). Interestingly, tissues on UV-M hydrogels reached a significantly higher OOP of 0.85±0.09 than the OOP of both UN and MM hydrogels (n=44 images, 4 slides), indicating a high degree of sarcomere alignment and organization. Therefore, UV laser micropatterning of gelatin hydrogels is a sufficient and promising tool for tissue engineering applications where sarcomeric alignment is required.

To further validate the translation of the rapid manufacturing method to human cell models, anisotropic cardiac tissues were engineered from human induced pluripotent stem cell-derived cardiomyocytes (iPSCs) on the UV-M hydrogels. MM and grooved UV-M substrates were engineered as previously described and seeded iPSCs onto these scaffolds. Using immunohistochemistry on the fixed tissue constructs, the iPSCs were shown to form aligned monolayers and express sarcomeric α-actinin on both MM and UV-M hydrogels (FIGS. 16E and 16F). Furthermore, human iPSCs seeded on UV-M gelatin remain viable for several days in culture (fixed at 9 days) and exhibit spontaneous contractions along the UV micropatterns at ˜1 beat per second.

To investigate cellular interactions with UV-μP single cell islands, iPSCs were seeded on these hydrogels and verified that cellular adhesion and sarcomeric α-actinin expression were in agreement with previous studies (FIGS. 16G and 16H) (Pasqualini. Stem Cell Reports, 2015 4(3): p. 340-347). Moreover, human iPSCs were found to respond to the μ-pillars in two distinct ways. In some cases, cells remained confined within the boundaries of a single pillar and assumed a spherical shape that was denoted as a ‘compact iPSC’ (FIG. 16G). Alternatively, ‘spread iPSCs’ expanded beyond a single pillar and aligned to one major axis, such that sarcomeric α-actinin is oriented around the nucleus of the cell where the central pillar is located (FIG. 16H).

Human iPSCs seeded on UV-μP gels were investigated to determine whether they exhibited sarcomeric organization in agreement with previous microcontact printing studies (supra). Sarcomeric packing density (SPD) of contractile proteins, like α-actinin, are a metric of the degree of sarcomeric organization and cellular maturation of single iPSCs (Id.). As detailed in the methods, the SPD is a scoring system for maturation of the iPSC cytoskeleton. A SPD score of 0 represents diffuse sarcomeric α-actinin staining and poor orientation, while a score of 1 represents a highly organized lattice of sarcomeric α-actinin. The SPD of human iPSCs seeded on the UV-μP gels exhibited an average of 0.22±0.01 (1 slide, n=8 images). This SPD value is in agreement with previously published experiments for human iPSCs seeded on microcontact printed islands where SPD is within a 0.1 to 0.3 range. This is typical of human iPSCs, as cellular maturation of the sarcomeric lattice structure is immature (Czerner. Procedia Materials Science, 2015) and contain heterogeneous populations of myocytes (Birket et al. Nat Biotech, 2015. 33(9): p. 970-979). Without being bound by any one particular theory, these results also suggest that in the future, UV laser micropatterning may aid in providing substrates for human iPSC single cell studies, including studies on 3-dimensional nuclear morphologies and cardiac contractile function of tissues (Bray. Biomaterials, 2010), contractility measurements of single cells using microposts (Rodriguez. Journal of Biomechanical Engineering, 2014; Fu. Nat Meth, 2010), and traction force microscopy techniques (Lee. The Use of Gelatin Substrates for Traction Force Microscopy in Rapidly Moving Cells, in Methods in Cell Biology. 2007, Academic Press. p. 295-312; Aratyn-Schaus et al. The Journal of Cell Biology, 2016 DOI: 10.1083/jcb.201508026).

### Heart-On-a-Chip Applications of UV Laser Micropatterning

To further advance UV laser micropatterning as a rapid fabrication method for heart-on-a-chip applications, this UV-laser patterning method was applied to fabricate an established heart on-a-chip design called the muscular thin film (MTF) assay that enables the quantitative readout of contractile stress in engineered microtissues (Feinberg. Science, 2007). Heart-on-a-chip MTFs consist of engineered cardiac muscle tissue on micropatterned cantilevers (McCain. Biomaterials, 2014). This is achieved by measuring how far muscle contraction lifts up a thin polymeric or hydrogel cantilever, which provides a quantitative readout of contractile stress [Feinberg. Science, 2007; Alford et al. Biomaterials, 2010. 31(13): p. 3613-21; Nesmith et al. The Journal of Cell Biology, 2016 DOI: 10.1083/jcb.201603111; Eric et al. Biofabrication, 2014. 6(4): p. 045005). As the muscle contracts, the cantilever bends, and the applied contractile stress can be computed from the cantilevers' curvature according to a modified Stoney's equation for deformation of stressed thin films (Lind et al. Nat Mater, 2016; Grosberg. Lab Chip, 2011].

Here, the contractile function of cardiac tissues engineered on UV-M hydrogels was investigated and a protocol to fabricate UV-M based MTFs (FIG. 1) was developed. then NRVMs were seeded onto these constructs (FIG. 16C) to generate aligned cardiac tissues. The UV laser was employed for patterning the microgrooves and cutting out the thin film cantilevers simultaneously (FIG. 6A and FIG. 1). Neonatal rat ventricular myocytes attached to the thin film cantilevers and exhibited spontaneous contractions in culture (FIG. 6Bi-ii, 6C). Custom tracking software was used to measure the x-projection of the thin film cantilevers during spontaneous and electrically paced contractions (FIG. 6B i-ii). These measurements were used to derive the films' curvature and corresponding contractile stress using a modified Stoney's equation for diastolic (FIG. 6B i) and systolic states (FIG. 6B ii). Previously measured bulk stiffness of 55 kPa was chosen for these calculations as opposed to the surface stiffness of the gelatin, as this is more relevant to the cantilever movement through the medium (Lind. Nat Mater, 2016; McCain. Biomaterials, 2014). From the raw stress measurements (FIG. 6B iii), the difference between diastolic and systolic stress as the twitch stress (FIG. 6B iv, gray bars) was quantified. During spontaneous contractions, UV-M MTFs exhibited average diastolic stresses of 24.7±0.6 kPa and average systolic contractile stresses of 26.5±0.7 kPa (n=12 films); these values are on the same order of magnitude as previously published for MM MTFs (McCain. Biomaterials, 2014). With electrical pacing, diastolic stresses of UV-M MTFs remained at 23.4±0.8 kPa at 1 Hz (n=13 films) and 24.1±0.7 kPa at 2 Hz (n=9 films) (FIG. 16 iv and Table I). The systolic stress during pacing remained near-constant at 26.5±1.0 kPa at 1 Hz and 27.3±1.2 kPa at 2 Hz. The average twitch stress increased non-significantly from 1.8±0.3 kPa for spontaneously contracting thin films to 3.1±0.4 kPa at 1 Hz and 3.3±0.5 kPa at 2 Hz pacing (Table I). This result is in agreement with previous studies in MM hydrogels (Id.) and confirms that UV-M gels are suitable scaffolds for measuring cardiac contractile function. To this end, UV-M hydrogels that support MTF technology without the need for soft lithography or mask design were fabricated.

The spontaneous beat rate of engineered NRVM tissues on UV-M and MM gels were compared over a 27 day period, as gelatin has been show in improved tissue viability and function for up to a month (Id.). NRVM tissues cultured on MM and UV-M gels exhibit similar beat rate patterns over the 27 day period (FIG. 6C). The beat rate for tissues cultured on MM gels from 1.5±0.3 beats per second (day 3, n=3 tissues) to 0.5±0.1 beats per second (day 27, n=3 tissues). The beat rate for tissues cultured on UV-M gels ranged from 1.2±0.3 beats per second (day 3, 5 films) to 0.7±0.5 beats per second (day 27, n=3 films).

The effect of long term culture on MTF contractile stress for UV-M tissues (FIG. 6D) was investigated. Tissues cultured on UV-M MTFs were paced at 1 and 2 Hz as previously described. Long term culture significantly reduced diastolic stress to 12.6±0.6 kPa at 1 Hz and 21.8±0.01 kPa at 2 Hz (n=2-3 films, 1 chip) compared to UV-M MTFs at 5 days in culture. Furthermore, UV-M MTFs cultured for 27 days exhibited systolic stresses comparable to tissues cultured for 5 days with mean systolic stress of 23.2±0.1 kPa at 2 Hz and significantly reduced systolic stress of 17.7±0.8 kPa at 1 Hz compared to tissues at 5 days at the same pacing rate (n=2-3 films, 1 chip, FIG. 6Biv and 6D). Interestingly, long term culture did not significantly alter the contractile twitch stresses at 1 or 2 Hz with mean stresses of 5.1±0.6 kPa and 1.5±0.2 kPa, respectively (n=2-3 films, 1 chip, Table I). This demonstrates that UV-M gelatin allows for the long term use of NRVM muscular thin films that can be adapted for more advanced heart-on-a-chip technologies.

In summary, these results show that the fabrication method of UV laser-mediated micropatterning of gelatin hydrogels allows for structural organization of sarcomeric α-actinin that is required to generate appropriate contractile responses on tissue-engineered muscular thin films. Furthermore, the ability to culture muscular thin films on the UV-M gels is demonstrated for a 27 day period, which makes them suitable for long-term studies on this platform. These results serve as the quality control metrics for effective cardiac tissue engineering that can be adapted to microfluidic heart-on-a-chip technologies in future studies.

### SUMMARY

A new UV-laser mediated photopatterning method for the automated and flexible top-down micropatterning of gelatin hydrogel films for tissue engineering application is demonstrated herein. This approach complements the current methods for patterning hydrogel substrates using stamps (Id.) or 3D-printing (Yanagawa et al. Regenerative Therapy, 2016. 3: p. 45-57), which are reliable and accurate techniques allowing for complex feature generation, but they are also costly, labor-intensive, and inflexible. In particular, a protocol for activating gelatin hydrogels with a non-toxic UV-photosensitizer, riboflavin-5′phosphate was developed, which allows for the subsequent photoablation of micropatterns into the surface using a UV laser engraver. Three key parameters of reliable pattern generation were identified and optimized: (1) the type and concentrations of gelatin photosensitizers; (2) UV laser parameters; (3) and choice of carrier substrate. Using this method, standard micropatterned substrates were designed and fabricated for use in cardiac tissue engineering that are more than two times faster, but at the same microscale spatial resolution and low variability, compared to traditional, manual bottom-up fabrication using photolithography and micromolding. Importantly, this photopatterning method does not modify the stiffness of the gelatin surface. In contrast, it was found that traditional micromolding of gelatin leads to a slight stiffening of the gel's surface compared to flat, homogeneous substrates, potentially by introducing stiffness-altering tensions during the cooling, drying and polymerization of the gelatin in the mold (Rizzieri et al. Langmuir, 2006. 22(8): p. 3622-3626). Hence, UV-patterning facilitates greater control of substrate stiffness, a major factor affecting cultured cell and tissue biology (Discher et al. Science, 2005. 310(5751): p. 1139-1143). The suitability of UV-patterned substrates for cardiac muscle engineering was validated using both primary neonatal rat cardiomyocytes and human induced pluripotent stem cell (iPSC)—derived cardiomyocytes. It is shown that, comparable to established MM substrates, the UV-M substrates support adhesion, alignment, contractile response, multi-week function, and viability of these cells types in culture.

The potential use of UV-M gelatin substrates is, however, not restricted to cardiac muscle chips. The engineering of other highly polarized and anisotropic organ tissues, such as neural tissue and skeletal muscle, equally benefit from micropatterned substrates (Verhulsel et al. Biomaterials, 2014. 35(6): p. 1816-1832). Topographically patterned surfaces can also be used to mimic tissue-tissue interfaces and evoke characteristic cellular behaviors at these boundaries such as altered cell adhesion, migration, proliferation and matrix deposition. This demonstrates that UV micropatterning has high applicability to organ chips by probing the dynamic interplay of mechanical forces and different cell types involved in forming healthy and diseased tissue interfaces (Nikkhah et al. Biomaterials, 2012. 33(21): p. 5230-5246; Hamilton et al. Calcified Tissue International, 2006. 78(5): p. 314-325; Ning et al. Langmuir, 2016. 32(11): p. 2718-2723).

The UV-patterning method allows separating the process of substrate fabrication and substrate patterning in both space and time. Such modular fabrication has a great potential to further increase throughput and flexibility because it enables batch processing, which reduces the relative cost of time-intensive start-up and calibration steps. Specifically, large quantities of gelatin films could be prepared using dedicated injection molding or spin-coating set-ups (Wilson et al. Lab on a Chip, 2011. 11(8): p. 1550-1555; Gitlin et al. Lab on a Chip, 2009. 9(20): p. 3000-3002; Scott et al. Physics World, 1998. 11(5): p. 31). Once dried, these samples can then be stored for “on-demand” patterning, eliminating the multi-day delay between pattern design and sample fabrication typical for micromolding and the associated photolithography steps (McCain, et al. Biomaterials, 2014; Whitesides. Annual Review of Biomedical Engineering, 2001; Scott. Physics World, 1998). Further, the UV-patterning step could be scaled and standardized for batch processing by using a motorized stage that moves a set of samples through the active laser zone, similar to an assembly line.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

1. A method, comprising:
(a) modifying a surface energy of at least a portion of a surface of a base comprising a cyclic olefin copolymer (COC);
(b) forming a hydrogel layer on the surface of the base overlying the portion of the surface having the modified surface energy, the hydrogel layer being susceptible to cross-linking by exposure to light, the hydrogel layer having a surface facing away from the base, wherein the modification of the surface energy of the portion of the surface of the base promotes adhesion of the hydrogel layer to the surface of the base; and
(c) exposing at least a portion of the hydrogel layer to light in a preselected pattern, thereby optically micropatterning the surface of the hydrogel layer.

2. The method of claim 1, wherein the surface energy of at least the portion of the surface of the base is modified by plasma treatment.

3. The method of claim 1, wherein the preselected pattern is an anisotropic pattern.

4. The method of claim 1, wherein the preselected pattern is a geometric shape.

5. The method of claim 4, wherein geometric shape is a square saw-tooth, a rectangle, a square, a circle, or a triangle.

6. The method of claim 1, wherein the pre-selected pattern includes a plurality of lines or a plurality of line segments with a peak-to-peak line separation in a range of 1 μm to 100 μm.

7.-9. (canceled)

10. The method of claim 1, wherein a peak-to-trough height of the resulting micropattern in the surface of the hydrogel layer falls in a range of 0.5 μm to 10 μm.

11.-13. (canceled)

14. The method of claim 1, wherein a laser is used to expose the portion of the hydrogel layer to light in the preselected pattern.

15. The method of claim 14, wherein exposing the portion of the hydrogel layer to light in the preselected pattern comprises serially writing the preselected pattern into the hydrogel layer using the laser.

16.-26. (canceled)

27. The method of claim 1, wherein the wavelength of the light is 315 nm to 380 nm.

28. The method of claim 27, wherein the wavelength of the light is 355 nm.

29. The method of claim 1, wherein forming the hydrogel layer on the surface of the base overlying the portion of the surface having the modified surface energy comprises depositing an aqueous solution comprising a hydrogel on the surface of the base.

30. The method of claim 29, wherein the aqueous solution further comprises transglutaminase.

31.-33. (canceled)

34. The method of claim 29, wherein forming the hydrogel layer on the surface of the base overlying the portion of the surface having the modified surface energy further comprises curing the deposited aqueous solution resulting in a cured layer.

35.-37. (canceled)

38. The method of claim 34, wherein forming the hydrogel layer on the surface of the base overlying the portion of the surface having the modified surface energy further comprises treating the cured layer with a second solution that makes the cured layer susceptible to cross-linking by exposure to light.

39. The method of claim 38, wherein the second solution comprises riboflavin-5′ phosphate, Rose Bengal, or SU-8 Photoresist.

40. The method of claim 39, wherein the second solution comprises riboflavin-5′ phosphate.

41. The method of claim 40, wherein the second solution comprises 0.01% w/v to 0.3% w/v riboflavin-5′ phosphate.

42. The method of claim 41, wherein the second solution comprises 0.05% w/v riboflavin-5′ phosphate.

43. The method of claim 41, wherein the second solution comprises 0.1% w/v riboflavin-5′ phosphate

44. (canceled)

45. The method of claim 38, wherein cured layer is hydrated in the aqueous solution prior to treating the cured layer with the second solution.

46.-48. (canceled)

49. The method of claim 45, wherein the method further comprises:
masking a portion of the surface of the base using an adhesive mask prior to step (a), wherein the surface energy of the masked portion of the surface of the base is not modified during the modification of the surface energy of at least a portion of the surface of the base; and
removing the adhesive mask from the surface of the base after hydration of the cured layer.

50. (canceled)

51. The method of claim 1, further comprising drying the formed hydrogel layer prior to exposing at least the portion of the hydrogel layer to the light in the preselected pattern.

52. The method of claim 1, further comprising cutting through a full thickness of the hydrogel layer using a laser after the surface of the hydrogel layer has been micropatterned.

53. The method of claim 1, further comprising ablating a portion of the hydrogel layer using a laser after the surface of the hydrogel layer has been micropatterned.

54. The method of claim 1, further comprising modifying a surface energy of a portion of the surface of the base surrounding the micropatterned hydrogel layer to inhibit cell adhesion to the surface of the base.

55. (canceled)

56. (canceled)

57. The method of claim 1, further comprising seeding the micropatterned surface of the hydrogel layer with cells.

58. A fluidic device comprising a base and a gelatin layer having a micropatterned surface prepared according to claim 1, wherein the micropatterned surface is configured to support growth of a functional muscle tissue.

59. The fluidic device of claim 58, further comprising a functional muscle tissue disposed on the gelatin layer.

60. The fluidic device of claim 59, wherein the functional muscle tissue comprises cells selected from the group consisting of cardiac muscle cells, ventricular cardiac muscle cells, atrial cardiac muscle cells, striated muscle cells, smooth muscle cells, and vascular smooth muscle cells and combinations thereof.

61. A method, comprising:
(a) modifying a surface energy of at least a portion of a surface of a base;
(b) depositing an aqueous solution comprising a hydrogel on the surface of the base, wherein said solution comprises transglutaminase;
(c) curing the deposited aqueous solution resulting in a cured layer;
(d) treating the cured layer with a second solution that makes the cured layer susceptible to cross-linking by exposure to light; and
(e) exposing at least a portion of the hydrogel layer to light in a preselected pattern, thereby optically micropatterning the surface of the hydrogel layer.

62. The method of claim 61, wherein the surface energy of at least the portion of the surface of the base is modified by plasma treatment.

63. The method of claim 61, wherein the hydrogel layer has a surface facing away from the base.

64. The method of claim 61, wherein the modification of the surface energy of the portion of the surface of the base promotes adhesion of the hydrogel layer to the surface of the base.

65. The method of claim 61, wherein the second solution comprises riboflavin-5′ phosphate, Rose Bengal, or SU-8 Photoresist.

66. The method of claim 61, wherein the second solution comprises riboflavin-5′ phosphate.

67. The method of claim 61, wherein the base comprising a cyclic olefin copolymer (COC).
